(19) United States
(12) Patent Application Publication
(10) Pub. No.: US 2024/0368627 A1
(43) Pub. Date: Nov. 7, 2024

(54) GENE THERAPY PRODUCTS FACILITATING BYSTANDER EFFECTS AND METHODS USING THE SAME

(71) Applicant: The University of North Carolina at Chapel Hill, Chapel Hill, NC (US)

(72) Inventors: Haiyan Fu, Durham, NC (US); Tierra Bobo, Mebane, NC (US)

(21) Appl. No.: 18/682,987

(22) PCT Filed: Aug. 11, 2022
(86) PCT No.: PCT/US2022/074791

§ 371 Date: Feb. 12, 2024

Related U.S. Application Data
(60) Provisional application No. 63/232,420, filed on Aug. 12, 2021.

(51) Int. Cl. C12N 15/86; A61K 48/00; C07K 14/47; C12N 9/10

(52) U.S. Cl. CPC C12N 15/86; A61K 48/005; C07K 14/47; C12N 9/1029; C12Y 203/01078; C12N 2750/14143; C12N 2830/50

(57) ABSTRACT

This invention relates to methods and compositions for gene therapy. In particular, the invention relates to compositions for enhancing delivery of therapeutic products to bystander cells that have not received the expression vector encoding the therapeutic product. The invention further relates to methods of treating disorders using the compositions of the invention.

### STATEMENT OF PRIORITY

This application claims the benefit of U.S. Provisional Application Ser. No. 63/232,420, filed Aug. 12, 2021, the entire contents of which are incorporated by reference herein.

### GOVERNMENT SUPPORT

This invention was made with government support under Grant Number NS118165 awarded by the National Institutes of Health. The government has certain rights in the invention.

### FIELD OF THE INVENTION

This invention relates to methods and compositions for gene therapy. In particular, the invention relates to compositions for enhancing delivery of therapeutic products to bystander cells that have not received the expression vector encoding the therapeutic product. The invention further relates to methods of treating disorders using the compositions of the invention.

### BACKGROUND OF THE INVENTION

Approximately 1 in 10 people in the US suffers from a rare genetic disease, which can seriously impact life-span, quality of life, independence, and economic potential. Gene therapy is the most promising form of treatment for the correction of heritable diseases. Among gene therapy delivery vehicles, adeno-associated virus (AAV) vectors have shown therapeutic effect in numerous clinical trials. Currently, 13 serotypes and numerous AAV variants and mutants have been isolated and studied as gene delivery vehicles. Several AAV serotypes, such as AAV2, AAV8, and AAV9, have been extensively employed in clinical trials and achieved therapeutic effects. Despite clinical success, emerging concerns about limited transduction efficacy and the high vector dose requirement remain crucial barriers for ongoing AAV-based gene therapy in preclinical and clinical settings.

Mucopolysaccharidosis (MPS) IIIC is a devastating ultra-rare lysosomal storage disease (LSD), with estimated incidence of 1 in 1,500,000 live births.¹ It is caused by autosomal recessive defects in the heparan alpha-glucosaminide N-acetyltransferase (HGSNAT) gene. HGSNAT is a transmembrane enzyme responsible for the acetylation of the non-reducing terminal alpha-glucoasmine residue of heparan sulfate (HS), a class of biologically important glycosaminoglycans (GAGs). The lack of HGSNAT activity results in HS accumulation in the lysosomes in virtually all organs, especially in the nervous system, resulting in severe progressive neuropathy. Infants with MPS IIIC appear normal at birth, but developmental delay and severe neurological deterioration begin to occur in early childhood, leading to premature death before the 3rd decade.^{1,2} Somatic manifestations do occur in all individuals, though milder than other MPS disorders.

There is an urgent unmet medical need for MPS IIIC. No treatments are currently available for MPS IIIC. Therapies have been limited to supportive care. Unlike most of the other lysosomal enzyme proteins, HGSNAT is an exclusively transmembrane protein that is insoluble in an aqueous environment and not secreted, therefore having no bystander effect, posing a particular challenge to therapeutic development.

Gene therapy targeting the root cause is ideal for treating MPS IIIC, if broadly delivered to the central nervous system (CNS) and peripheral tissues, because of the potential for long-term endogenous production of recombinant enzymes. Among the gene delivery strategies, recombinant adeno-associated viral (rAAV) vector is an ideal tool for this application because it is safe with demonstrated long-term expression in the CNS and periphery.³ The demonstrated trans-BBB-neurotropic AAV93-5 has offered a great gene delivery tool for the treatment of monogenic diseases with neurological manifestations.⁶⁻¹⁰

Spinal muscular atrophy (SMA) is an autosomal recessive disorder of spinal motor neuron (SMN) protein deficiency, characterized by severe muscle atrophy due to the degeneration and loss of lower motor neurons (MN).¹¹⁻¹⁴ SMA has an incidence of 1:10,000 live births and is a common genetic cause of infant death.¹⁵⁻¹⁷ SMA is caused by a deletion or mutation in the survival motor neuron 1 (SMN1) gene and retention of the SMN2 gene, which results in the loss or reduced levels of SMN protein.¹⁸⁻²⁰ SMN is an essential protein and the loss of SMN is lethal in all species. Both SMN1 and SMN2 genes express SMN protein, and there are two forms of SMN. While SMN1 is the primary gene responsible for the functional production of SMN protein, the SMN2 gene contains a splice modulator that silences exon 7 and predominantly (90%) produces truncated unstable SMN protein (SMNΔ7).²¹⁻²⁴ Approximately 95-98% of affected individuals have deletions in the SMN1 gene and 2-5% have specific mutations in the SMN1 gene that result in a decreased production of the SMN protein. When three or more copies of the SMN2 gene are also present, the disease may be milder. Clinically, SMA is classified into 5 types based on the severity and onset of symptoms.^{12,21,25,26} SMA1 is the most common and severe form of the disease with marked MN degeneration, including MN loss in the ventral horn in the spinal cord and loss of ventral root axons in patients.²⁷⁻²⁹ Children with SMA1 have disease onset during infancy that results in failure to achieve motor milestones, leading to the need for mechanical ventilation before 2 years of age and premature death.³⁰

Before 2017, therapies for SMA were limited to case management and supportive care. However, SMA therapeutic research and development efforts have enabled recent breakthrough advancements, leading to FDA approval of nusinersen in 2017, zolgensma in 2019, and risdiplam in 2020, for the treatment of SMA. Both nusinersen (an antisense oligonucleotide drug administered intrathecally) 31-34 and risdiplam (an oral small molecule drug) 35-37 modify the splicing of SMN2 mRNA, leading to an increase in functional SMN levels and requiring routine administration. Zolgensma is a gene therapy drug, replacing the defective SMN1 gene via a single systemic delivery using an adeno-associated viral serotype 9 (AAV9) vector, with the potential for long-term benefits.^{3,6} These drugs offer significant functional benefits and have fundamentally changed the clinical landscape of SMA, though they have not provided a cure. While approved, the most critical issue in zolgensma clinical application is the requirement for very high vector doses, because it needs to transduce as many cells as possible to achieve optimal benefits. Notably, the high dose requirement of zolgensma challenges the scale-up capacity of AAV manufacturing. This challenge will be more critical when considering the potential of using zolgensma to treat juvenile and adult patients with SMA (types 2-4). While intrathecal delivery may require much lower vector doses than the systemic route, studies of the antisense oligonucleotide drug nusinersen showed that peripheral SMN restoration is also essential for long-term rescue of severe SMA.³⁸

Extracellular vesicles (EVs) are membrane-bound vesicles released from cells, including plasma-membrane-derived ectosomes (100 nm-1000 nm) and exosomes of endocytic origin (30 nm-150 nm).^{39,40} EVs have been demonstrated to facilitate intercellular communication and signaling to recipient cells by all cell types.^{40,41} All cell types continuously release EVs to the extracellular environment, including the blood and virtually all bodily fluids.

Interestingly, EVs carry lipids, proteins, mRNA, microRNAs (miRNAs), genomic DNA, and mitochondrial DNA.³⁹⁻⁴¹ All cells are now known to communicate by the exchange of large molecules via EV traffic.^{40,42} Once released, EVs bind to neighboring cells, or to the extracellular matrix, or traffic via the blood circulation or other body fluids. EVs participate in important biological functions as mediators of intercellular communication by adhering to the surface of recipient cells or being internalized by recipient cells, and releasing genetic content into recipient cells.⁴³⁻⁴⁵ Once in the recipient cell, EV RNAs are functional as they would be in the originating source cells.⁴⁶⁻⁵⁰ Given that the EV “cargo” can be delivered to other cells, loaded EVs or engineered EVs have been used as vehicle in drug delivery studies.^{53,52} Further, the presence of EVs has been linked to pathological conditions, such as autoimmune diseases including multiple sclerosis (MS) and cancer, in which increasing concentrations of EVs have been reported.⁵³⁻⁵⁵ EVs have been implicated in neurogenesis, synaptic activities, and normal functioning of the nervous system, and therefore have been used as vehicles in therapeutic studies for treating neurological diseases,⁵⁶ possibly including the ability to cross the blood-brain-barrier (BBB).

While the mechanisms governing how specific contents are packaged into EVs remain poorly understood, previous studies have shown that RNAs packaged in EVs include mRNA, miRNA, snRNA, lncRNA, rRNA, and tRNA.^{57,58} Notably, studies by Bolukbasi et al. provide a clue as to how genetic material are uploaded into EVs, in that they identified a 25 nucleotide (nt) “zip code” (ZC) sequence in the 3′-untranslated regions (3′-UTR) of mRNAs.⁵⁹ Importantly, they further demonstrated that incorporation of this 25 nt sequence into the 3′-UTR of a reporter gene in a plasmid led to the enrichment of reporter mRNA in EVs and a significant increase in reporter gene expression in recipient cells.⁵⁹ Further, recent studies indicate that numerous RNA binding proteins (RBPs) are transported into exosomes in association with RNA molecules in the form of RNA-RBP complexes with both cellular RNA and exosomal-RNA species.⁶⁰⁻⁶⁵ This type of association could be a general mechanism for RNA transport and maintenance in exosomes, and may favor the shuttling of RNAs from exosomes to recipient cells in the form of stable complexes.⁶⁰⁻⁶⁵

The present invention overcomes shortcomings in the art by providing novel methods and compositions for facilitating bystander effects via extracellular vesicles, permitting effective therapy with lower doses of vectors.

### SUMMARY OF THE INVENTION

The present invention is based on the development of gene therapy vectors that enhance delivery of therapeutic gene products to bystander cells that did not receive the vector. The vectors facilitate the efficient expression of the gene products in bystander cells by providing abundant EVs containing mRNA produced from the gene and effective delivery of the mRNA and expression of the protein in bystander cells. The invention is effective for both secreted and non-secreted proteins, but may be especially effective for non-secreted proteins where a bystander effect from the protein being secreted from transduced cells cannot occur and enzyme replacement therapy is not a viable option. The invention strongly supports the therapeutic potential of EV-facilitated bystander effects of gene replacement therapy. Further, the EV-facilitated bystander effects have a great potential for reducing the burden of scale-up vector production for treating diseases in humans.

Thus, one aspect of the invention relates to an expression vector comprising a polynucleotide encoding a nucleic acid of interest operably linked to an extracellular vesicle-targeting zip code sequence and a virus particle and pharmaceutical composition comprising the expression vector.

A further aspect of the invention relates to methods of delivering a nucleic acid of interest to bystander cells in a subject, comprising administering to the subject an effective amount of the expression vector, virus particle, or pharmaceutical composition of the invention, thereby forming extracellular vesicles comprising the nucleic acid of interest and delivering the nucleic acid of interest to bystander cells.

An additional aspect of the invention relates to methods of treating a disorder treatable by expression of a nucleic acid of interest in a subject in need thereof, comprising administering to the subject a therapeutically effective amount of the expression vector, virus particle, or pharmaceutical composition of the invention, thereby treating the disorder.

These and other aspects of the invention are set forth in more detail in the description of the invention below.

### BRIEF DESCRIPTION OF THE DRAWINGS

FIG. 1 shows the schematic structure of the rAAV-hHGSNAT vector genome. ITR: AAV2 inverted terminal repeats; hHGSNAT: human HGSNAT cDNA; CMV: human CMV immediate early enhancer/promoter; ZC: 25 nt EV zip-code signal; Poly A: SV40 poly A.

FIGS. 2A-2B show AAV-mediated rHGSNAT expression in vitro in human MPS IIIC cells. GM05157 cells in 10 cm plates were transfected in duplicate with the AAV-hHGSNAT vector plasmids (A), or infected with rAAV2-hHGSNAT viral vectors (B). 48 h later, cell lysates were assayed in duplicate for HGSNAT activity and for hHGSNAT mRNA by qRT-PCR. HGSNAT activity: units/mg protein, 1 unit=mmol 4 MU released/hour. qRT-PCR data is expressed as fold of change (2\hat{ }ddCT) vs. untransduced cells. Healthy: GM00969 cells; NT: non-treated GM05157 cells; HGSNAT: GM05157 transfected or infected with AAV-HGSNAT vectors; ZC: GM05157 transfected or infected with AAV-HGSNAT^{zc} vectors. \hat{ }: P<0.05 vs. NT; *: P<0.05 vs. Healthy; #: P<0.05 vs. ZC. Data were from 2 sets of experiments each in duplicate, and each sample was assayed in duplicate.

FIGS. 3A-3B show AAV-mediated HGSNAT mRNA in EVs from human MPS IIIC cells. 48 h post vector plasmid transfection or rAAV2 vector infection in GM05157 cells, media were processed to isolate EVs by ultracentrifugation. RNA extracted from EVs were assayed in duplicate by qRT-PCR for hHGSNAT mRNA. Utx: EVs from untreated GM05157; HGSNAT: EVs from GM05157 transfected or infected with HGSNAT vectors; ZC: GM05157 transfected or infected with HGSNAT^{zc} vectors. \hat{ }: P<0.05 vs. NT; #: P<0.05 vs. ZC. Data were from 2 sets of experiments.

FIGS. 4A-4B show characterization of EVs. 48 h after plasmid transfection or rAAV2 infection in GM05157 cells, media samples were processed to isolate EVs by ultracentrifugation. The isolated EVs were analyzed using NanoSight NS500. Healthy: EVs from GM00969 cells; Utx: EVs from untreated GM05157 cells; HGSNAT: EVs from GM05157 cells transfected or infected with HGSNAT vectors; ZC: EVs from GM05157 transfected or infected with HGSNATzc vectors. *: EVs data from 2 sets of transfections; **: data on combined EVs from 2 sets of infection.

FIGS. 5A-5B show EV-facilitated by-stander effect in MPS IIIC cells. 48 h after plasmid transfection or rAAV2 infection in GM05157 cells, media samples were processed to isolate EVs by ultracentrifugation. The isolated EVs were added to the media of GM05157 cells, and 48 h after incubation, the cell lysates were assayed for HGSNAT activity. Utx: GM05157 cells incubated with EVs from untreated MPS IIIC cells; HGSNAT: GM05157 cells incubated with EVs from GM05157 cells transfected or infected with HGSNAT vectors; ZC: GM05157 cells incubated with EVs from GM05157 cells transfected or infected with HGSNAT^{zc} vectors.

FIGS. 6A-6B show AAV-mediated correction of GAG storage in vitro in human MPS IIIC cells. GM05157 cells in 10 cm plates were transfected in duplicate with ptr-CMV-hHGSNAT or ptr-CMV-hHGSNAT^{zc} plasmids (7 μg/plate). 48 h later, cell lysates were assayed in duplicate for GAG contents (A). Media from these transfected cells were applied to non-treated GM05157 cells, and after 48 h incubation, cell lysates were assayed in duplicate for GAG contents (B). GAG contents are expressed as μg/5×10⁵ cells. HC: healthy human fibroblasts (GM00969) cells; NT: non-treated GM05157 cells; ZC: GM05157 transfected with hHGSNAT^{zc} plasmid or incubated with media of hHGSNAT^{zc} transfected cells.

FIG. 7 shows the schematic structure of the scAAV-hHGSNAT vector genome. ITR: wt AAV2 inverted terminal repeats; dTR: AAV2 terminal repeat with deletion of terminal resolution site; hHGSNAT: human HGSNAT cDNA; mCMV: truncated miniature CMV promoter; ZC: 25nt EV zip-code signal; Poly A: SNRP-1 poly A signal.

FIG. 8 shows generation of scAAV-mCMV-hHGSNAT vectors. HEK293 cells were co-transfected by ptr-CMV-hHGSNAT, ptr-CMV-hHGSNAT^{ZC}, ptrs-mCMV-hHGSNAT, or ptrs-mCMV-hHGSNAT^{ZC}, and the helper plasmids pHELP and pAAV2/9, to generate single-stranded rAAV vectors and scAAV vectors. Purified AAV9 vector products were analyzed by alkaline denaturing gel electrophoresis. rAAV: single-stranded AAV vectors; scAAV: self-complementary AAV vectors.

FIG. 9 shows the schematic structure of the scAAV-CMV-hSMN1 vector genome. ITR: AAV2 inverted terminal repeats; dTR: AAV2 terminal repeat with deletion of terminal resolution site; CMV: CMV immediate early enhancer/promoter; hSMN1: human SMN1 coding sequence cDNA; ZC: 25 nt EV zip-code signal; Poly A: SV40 poly A signal.

FIGS. 10A-10D show AAV-mediated rSMN1 expression in vitro. Human SMA1 skin fibroblast cells (GM00232) in 10 cm plates were transfected in duplicate with the scAAV-hSMN1 vector plasmids. 48 h later, cells samples were assayed for SMN expression (SMN/GEM1) by western blot (A) and by qRT-PCR (B). Media of transfected cells were processed to extract EVs. Extracted EVs were added to the growth media of non-treated GM00232 cell cultures, and after 24 h incubation cell samples were assayed for SMN expression (SMN/GEM1) by western blot (C) and by qRT-PCR (D). NT: Non-treated GM00232 cells; SMN: GM00232 transfected with AAV-hSMN1 vector or incubated with EVs from AAV-hSMN1-transfected GM00232 cells; SMNZC: GM00232 transfected with AAV-hSMN1^{zc} vector or incubated with EVs from AAV-hSMN1^{zc}-transfected GM00232 cells.

### DETAILED DESCRIPTION OF EMBODIMENTS OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Except as otherwise indicated, standard methods known to those skilled in the art may be used for production of recombinant and synthetic polypeptides, antibodies or antigen-binding fragments thereof, manipulation of nucleic acid sequences, production of transformed cells, the construction of rAAV constructs, modified capsid proteins, packaging vectors expressing the AAV rep and/or cap sequences, and transiently and stably transfected packaging cells. Such techniques are known to those skilled in the art. See, e.g., SAMBROOK et al., MOLECULAR CLONING: A LABORATORY MANUAL 4th Ed. (Cold Spring Harbor, NY, 2012); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

### General Definitions

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together.

The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

The term “tropism” as used herein refers to preferential but not necessarily exclusive entry of the vector (e.g., virus vector) into certain cell or tissue type(s) and/or preferential but not necessarily exclusive interaction with the cell surface that facilitates entry into certain cell or tissue types, optionally and preferably followed by expression (e.g., transcription and, optionally, translation) of sequences carried by the vector contents (e.g., viral genome) in the cell, e.g., for a recombinant virus, expression of the heterologous nucleotide sequence(s).

The term “tropism profile” refers to the pattern of transduction of one or more target cells, tissues and/or organs. Representative examples of chimeric AAV capsids have a tropism profile characterized by efficient transduction of cells of the central nervous system (CNS) with only low transduction of peripheral organs (see e.g., U.S. Pat. No. 9,636,370 McCown et al., and US patent publication 2017/0360960 Gray et al.). Vectors (e.g., virus vectors, e.g., AAV capsids) expressing specific tropism profiles may be referred to as “tropic” for their tropism profile, e.g., neuro-tropic, liver-tropic, etc.

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the term “polypeptide” encompasses both peptides and proteins, unless indicated otherwise.

A “polynucleotide,” “nucleic acid,” or “nucleotide sequence” may be of RNA, DNA or DNA-RNA hybrid sequences (including both naturally occurring and non-naturally occurring nucleotides), but is preferably either a single or double stranded DNA sequence.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc. The region in a nucleic acid sequence or polynucleotide in which one or more regulatory elements are found may be referred to as a “regulatory region.”

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

The term “open reading frame (ORF),” as used herein, refers to the portion of a polynucleotide (e.g., a gene) that encodes a polypeptide, and is inclusive of the initiation start site (i.e., Kozak sequence) that initiates transcription of the polypeptide. The term “coding region” may be used interchangeably with open reading frame.

The term “codon-optimized,” as used herein, refers to a gene coding sequence that has been optimized to increase expression by substituting one or more codons normally present in a coding sequence with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry and/or initiation sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell. Codon-optimization also provides the ability to distinguish a codon-optimized gene and/or corresponding mRNA from an endogenous gene and/or corresponding mRNA in vitro or in vivo.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

As used herein, the term “modified,” as applied to a polynucleotide or polypeptide sequence, refers to a sequence that differs from a wildtype sequence due to one or more deletions, additions, substitutions, or any combination thereof.

As used herein, by “isolate” (or grammatical equivalents) a virus vector, it is meant that the virus vector is at least partially separated from at least some of the other components in the starting material.

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

As used herein, the term “prevent,” “prevents,” or “prevention” (and grammatical equivalents thereof) means to delay or inhibit the onset of a disease. The terms are not meant to require complete abolition of disease, and encompass any type of prophylactic treatment to reduce the incidence of the condition or delays the onset of the condition.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

A “heterologous nucleotide sequence” or “heterologous nucleic acid,” with respect to a virus, is a sequence or nucleic acid, respectively, that is not naturally occurring in the virus. Generally, the heterologous nucleic acid or nucleotide sequence comprises an open reading frame that encodes a polypeptide and/or a nontranslated RNA.

A “vector” refers to a compound used as a vehicle to carry foreign genetic material into another cell, where it can be replicated and/or expressed. A vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene/ORF. Insertion of a vector into the target cell is referred to as transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the term “viral vector” and “delivery vector” (and similar terms) in a specific embodiment generally refers to a virus particle that functions as a nucleic acid delivery vehicle, and which comprises the viral nucleic acid (i.e., the vector genome) packaged within the virion. Viral vectors according to the present invention may include chimeric AAV capsids according to the invention and can package an AAV or rAAV genome or any other nucleic acid including viral nucleic acids. Alternatively, in some contexts, the terms “viral vector” and “delivery vector” (and similar terms) may be used to refer to the vector genome (e.g., vDNA) in the absence of the virion and/or to a viral capsid that acts as a transporter to deliver molecules tethered to the capsid or packaged within the capsid.

A “functional fragment” of a polypeptide or protein, as used herein, means a portion of a larger polypeptide that substantially retains at least one biological ability.

As used herein, the term “derivative” is used to refer to a polypeptide which differs from a naturally occurring protein or a functional fragment by minor modifications to the naturally occurring polypeptide, but which substantially retains the biological activity of the naturally occurring protein. Minor modifications include, without limitation, changes in one or a few amino acid side chains, changes to one or a few amino acids (including deletions, insertions, and/or substitutions) (e.g., less than about 20, 15, 10, 9, 8, 7, 6, 5, 4, 3, or 2 changes), changes in stereochemistry of one or a few atoms (e.g., D-amino acids), and minor derivatizations, including, without limitation, methylation, glycosylation, phosphorylation, acetylation, myristoylation, prenylation, palmitation, amidation, and addition of glycosylphosphatidyl inositol.

The term “substantially retains,” as used herein, refers to a fragment, derivative, or other variant of a polypeptide that retains at least about 50% of the activity of the naturally occurring polypeptide (e.g., binding to an antibody), e.g., about 60%, 70%, 80%, 90% or more.

The term “template” or “substrate” is used herein to refer to a polynucleotide sequence that may be replicated to produce the viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

### Expression Vectors

One aspect of the present invention relates to an expression vector comprising a polynucleotide encoding a nucleic acid of interest operably linked to an extracellular vesicle-targeting zip code sequence.

In some embodiments, the expression vector may be any nucleic acid delivery vector, e.g., a viral vector or a non-viral vector. In some embodiments, the viral vector is an adeno-associated virus, retrovirus, lentivirus, poxvirus, alphavirus, baculovirus, vaccinia virus, herpes virus, Epstein-Barr virus, or adenovirus vector. In some embodiments, the non-viral vector is a plasmid, liposome, electrically charged lipid, nucleic acid-protein complex, or biopolymer.

In some embodiments of the invention, the expression vector is a parvovirus vector. The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See, e.g., FIELDS et al., VIROLOGY, volume 2, chapter 69 (4th ed., Lippincott-Raven Publishers).

In some embodiments of the invention, the expression vector is a parvovirus within the genus Dependovirus. The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIELDS et al., VIROLOGY, volume 2, chapter 69 (4th ed., Lippincott-Raven Publishers); and Table 1. A number of additional AAV serotypes and clades have been identified (see, e.g., Gao et al., (2004) J. Virol. 78:6381-6388 and Table 1), which are also encompassed by the term “AAV.”

In some embodiments, the parvovirus vector is a self-complementary AAV vector.

As discussed above, the parvovirus particles and genomes of the present invention can be from, but are not limited to, AAV. The genomic sequences of various serotypes of AAV and the autonomous parvoviruses, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching parvovirus and AAV nucleic acid and amino acid sequences. See also, e.g., Bantel-Schaal et al., (1999) J. Virol. 73:939; Chiorini et al., (1997) J. Virol. 71:6823; Chiorini et al., (1999) J. Virol. 73:1309; Gao et al., (2002) Proc. Nat. Acad. Sci. USA 99:11854; Moris et al., (2004) Virol. 33-: 375-383; Mori et al., (2004) Virol. 330:375; Muramatsu et al., (1996) Virol. 221:208; Ruffing et al., (1994) J. Gen. Virol. 75:3385; Rutledge et al., (1998) J. Virol. 72:309; Schmidt et al., (2008) J. Virol. 82:8911; Shade et al., (1986) J. Virol. 58:921; Srivastava et al., (1983) J. Virol. 45:555; Xiao et al., (1999) J. Virol. 73:3994; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, PA (incorporated herein it its entirety).

The term “AAV viral vectors” includes “chimeric” AAV nucleic acid capsid coding sequence or AAV capsid protein is one that combines portions of two or more capsid sequences. A “chimeric” AAV virion or particle comprises a chimeric AAV capsid protein.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged. The virus vectors of the invention can further be “targeted” virus vectors (e.g., having a directed tropism) and/or a “hybrid” parvovirus (i.e., in which the viral ITRs and viral capsid are from different parvoviruses) as described in international patent publication WO 00/28004 and Chao et al., (2000) Mol. Therapy 2:619.

TABLE 1

| AAV | GenBank | AAV | GenBank | AAV | GenBank |
|---|---|---|---|---|---|
| Serotypes/ | Accession | Serotypes/ | Accession | Serotypes/ | Accession |
| Isolates | Number | Isolates | Number | Isolates | Number |
| Clonal Isolates | | Hu S17 | AY695376 | Cy3 | AY243019 |
| Avian AAV ATCC | AY186198, | Hu T88 | AY695375 | Cy5 | AY243017 |
| VR-865 | AY629583, | | | | |
| | NC_004828 | | | | |
| Avian AAV strain | NC_006263, | Hu T71 | AY695374 | Rh13 | AY243013 |
| DA-1 | AY629583 | | | | |
| Bovine AAV | NC_005889, | Hu T70 | AY695373 | | |
| | AY388617 | | | | |
| AAV4 | NC_001829 | Hu T40 | AY695372 | Clade E | |
| AAV5 | AY18065, | Hu T32 | AY695371 | Rh38 | AY530558 |
| | AF085716 | | | | |
| Rh34 | AY243001 | Hu T17 | AY695370 | Hu66 | AY530626 |
| Rh33 | AY243002 | Hu LG15 | AY695377 | Hu42 | AY530605 |
| Rh32 | AY243003 | | | Hu67 | AY530627 |
| AAV10 | AY631965 | Clade C | | Hu40 | AY530603 |
| AAV11 | AY631966 | AAV 3 | NC_001729 | Hu41 | AY530604 |
| AAV12 | DQ813647 | AAV 3B | NC_001863 | Hu37 | AY530600 |
| AAV13 | EU285562 | Hu9 | AY530629 | Rh40 | AY530559 |
| | | Hu10 | AY530576 | Rh2 | AY243007 |
| Clade A | | Hu11 | AY530577 | Bb1 | AY243023 |
| AAV1 | NC_002077, | Hu53 | AY530615 | Bb2 | AY243022 |
| | AF063497 | | | | |
| AAV6 | NC_001862 | Hu55 | AY530617 | Rh10 | AY243015 |
| Hu.48 | AY530611 | Hu54 | AY530616 | Hu17 | AY530582 |
| Hu 43 | AY530606 | Hu7 | AY530628 | Hu6 | AY530621 |
| Hu 44 | AY530607 | Hu18 | AY530583 | Rh25 | AY530557 |
| Hu 46 | AY530609 | Hu15 | AY530580 | Pi2 | AY530554 |
| | | Hu16 | AY530581 | Pi1 | AY530553 |
| Clade B | | Hu25 | AY530591 | Pi3 | AY530555 |
| Hu19 | AY530584 | Hu60 | AY530622 | Rh57 | AY530569 |
| Hu20 | AY530586 | Ch5 | AY243021 | Rh50 | AY530563 |
| Hu23 | AY530589 | Hu3 | AY530595 | Rh49 | AY530562 |
| Hu22 | AY530588 | Hu1 | AY530575 | Hu39 | AY530601 |
| Hu24 | AY530590 | Hu4 | AY530602 | Rh58 | AY530570 |
| Hu21 | AY530587 | Hu2 | AY530585 | Rh61 | AY530572 |
| Hu27 | AY530592 | Hu61 | AY530623 | Rh52 | AY530565 |
| Hu28 | AY530593 | | | Rh53 | AY530566 |
| Hu29 | AY530594 | Clade D | | Rh51 | AY530564 |
| Hu63 | AY530624 | Rh62 | AY530573 | Rh64 | AY530574 |
| Hu64 | AY530625 | Rh48 | AY530561 | Rh43 | AY530560 |
| Hu13 | AY530578 | Rh54 | AY530567 | AAV8 | AF513852 |
| Hu56 | AY530618 | Rh55 | AY530568 | Rh8 | AY242997 |
| Hu57 | AY530619 | Cy2 | AY243020 | Rh1 | AY530556 |
| Hu49 | AY530612 | AAV7 | AF513851 | | |
| Hu58 | AY530620 | Rh35 | AY243000 | Clade F | |
| Hu34 | AY530598 | Rh37 | AY242998 | AAV9 (Hu14) | AY530579 |
| Hu35 | AY530599 | Rh36 | AY242999 | Hu31 | AY530596 |
| AAV2 | NC_001401 | Cy6 | AY243016 | Hu32 | AY530597 |
| Hu45 | AY530608 | Cy4 | AY243018 | | |
| Hu47 | AY530610 | | | | |
| Hu51 | AY530613 | | | | |
| Hu52 | AY530614 | | | | |
| Hu T41 | AY695378 | | | | |

The AAV viral vectors of the invention may include a recombinant AAV vector genome. A “recombinant AAV vector genome” or “rAAV genome” is an AAV genome (i.e., vDNA) that comprises at least one inverted terminal repeat (e.g., one, two or three inverted terminal repeats) and one or more heterologous nucleotide sequences. rAAV vectors generally retain the 145 base terminal repeat(s) (TR(s)) in cis to generate virus; however, modified AAV TRs and non-AAV TRs including partially or completely synthetic sequences can also serve this purpose. All other viral sequences are dispensable and may be supplied in trans (Muzyczka, (1992) Curr. Topics Microbiol. Immunol. 158:97). The rAAV vector optionally comprises two TRs (e.g., AAV TRs), which generally will be at the 5′ and 3′ ends of the heterologous nucleotide sequence(s), but need not be contiguous thereto. The TRs can be the same or different from each other. The vector genome can also contain a single ITR at its 3′ or 5′ end. The terms “rAAV particle” and “rAAV virion” are used interchangeably here. A “rAAV particle” or “rAAV virion” comprises a rAAV vector genome packaged within an AAV capsid.

The term “terminal repeat” or “TR” includes any viral terminal repeat or synthetic sequence that forms a hairpin structure and functions as an inverted terminal repeat (ITR) (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or the SV40 hairpin that serves as the origin of SV40 replication can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

Parvovirus genomes have palindromic sequences at both their 5′ and 3′ ends. The palindromic nature of the sequences leads to the formation of a hairpin structure that is stabilized by the formation of hydrogen bonds between the complementary base pairs. This hairpin structure is believed to adopt a “Y” or a “T” shape. See, e.g., FIELDS et al., VIROLOGY, volume 2, chapters 69 & 70 (4th ed., Lippincott-Raven Publishers).

An “AAV terminal repeat” or “AAV TR” may be from any AAV, including but not limited to serotypes 1, 2, 3, 4, 5, 6, 7, 8, 9, 10 or 11 or any other AAV now known or later discovered (see, e.g., Table 1). An AAV terminal repeat need not have the native terminal repeat sequence (e.g., a native AAV TR sequence may be altered by insertion, deletion, truncation and/or missense mutations), as long as the terminal repeat mediates the desired functions, e.g., replication, virus packaging, integration, and/or provirus rescue, and the like.

Further, the viral capsid or genomic elements can contain other modifications, including insertions, deletions and/or substitutions.

As used herein, parvovirus or AAV “Rep coding sequences” indicate the nucleic acid sequences that encode the parvoviral or AAV non-structural proteins that mediate viral replication and the production of new virus particles. The parvovirus and AAV replication genes and proteins have been described in, e.g., FIELDS et al., VIROLOGY, volume 2, chapters 69 & 70 (4th ed., Lippincott-Raven Publishers).

The “Rep coding sequences” need not encode all of the parvoviral or AAV Rep proteins. For example, with respect to AAV, the Rep coding sequences do not need to encode all four AAV Rep proteins (Rep78, Rep 68, Rep52 and Rep40), in fact, it is believed that AAV5 only expresses the spliced Rep68 and Rep40 proteins. In representative embodiments, the Rep coding sequences encode at least those replication proteins that are necessary for viral genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40 proteins, Rep78 and Rep52 proteins, or Rep78 and Rep40 proteins.

As used herein, the term “large Rep protein” refers to Rep68 and/or Rep78. Large Rep proteins of the claimed invention may be either wildtype or synthetic. A wildtype large Rep protein may be from any parvovirus or AAV, including but not limited to serotypes 1, 2, 3a, 3b, 4, 5, 6, 7, 8, 9, 10, 11, or 13, or any other AAV now known or later discovered (see, e.g., Table 1). A synthetic large Rep protein may be altered by insertion, deletion, truncation and/or missense mutations.

Those skilled in the art will further appreciate that it is not necessary that the replication proteins be encoded by the same polynucleotide. For example, for MVM, the NS-1 and NS-2 proteins (which are splice variants) may be expressed independently of one another. Likewise, for AAV, the p19 promoter may be inactivated and the large Rep protein(s) expressed from one polynucleotide and the small Rep protein(s) expressed from a different polynucleotide. Typically, however, it will be more convenient to express the replication proteins from a single construct. In some systems, the viral promoters (e.g., AAV p19 promoter) may not be recognized by the cell, and it is therefore necessary to express the large and small Rep proteins from separate expression cassettes. In other instances, it may be desirable to express the large Rep and small Rep proteins separately, i.e., under the control of separate transcriptional and/or translational control elements. For example, it may be desirable to control expression of the large Rep proteins, so as to decrease the ratio of large to small Rep proteins. In the case of insect cells, it may be advantageous to down-regulate expression of the large Rep proteins (e.g., Rep78/68) to avoid toxicity to the cells (see, e.g., Urabe et al., (2002) Human Gene Therapy 13:1935).

As used herein, the parvovirus or AAV “cap coding sequences” encode the structural proteins that form a functional parvovirus or AAV capsid (i.e., can package DNA and infect target cells). Typically, the cap coding sequences will encode all of the parvovirus or AAV capsid subunits, but less than all of the capsid subunits may be encoded as long as a functional capsid is produced. Typically, but not necessarily, the cap coding sequences will be present on a single nucleic acid molecule.

The capsid structure of autonomous parvoviruses and AAV are described in more detail in BERNARD N. FIELDS et al., VIROLOGY, volume 2, chapters 69 & 70 (4th ed., Lippincott-Raven Publishers).

The extracellular vesicle-targeting zip code sequence may be any nucleotide sequence that increases the amount of mRNA produced from the nucleic acid of interest that is present in EVs and/or increases the percentage of EVs that contain the mRNA. In some embodiments, the extracellular vesicle-targeting zip code sequence, comprises, consists essentially of, or consists of the sequence of SEQ ID NO: 1 or a sequence at least 70% identical thereto, e.g., at least 75%, 80%, 85%, 90%, 95%, or 98% identical thereto.

The extracellular vesicle-targeting zip code sequence may be located in any position in the polynucleotide in which it is effective to target the mRNA to EVs. In some embodiments, the extracellular vesicle-targeting zip code sequence is linked to the 3′ end of the polynucleotide. In some embodiments, the polynucleotide is operably linked to a promoter and a poly (A) signal and the extracellular vesicle-targeting zip code sequence is located between the polynucleotide and the poly (A) signal.

In some embodiments, the expression vector is a plasmid that can be used to produce AAV vectors. In some embodiments, the expression vector comprises the sequence of any one of SEQ ID NOS: 2, 4, or 6 or a sequence at least 70% identical thereto, e.g., at least 75%, 80%, 85%, 90%, 95%, or 98% identical thereto.

Another aspect of the invention relates to a virus particle comprising the expression vector of the invention. In some embodiments, the virus particle is an AAV particle, an adenovirus particle, a herpesvirus particle, or a baculovirus particle.

In some embodiments, the nucleic acid of interest encodes a protein or nucleic acid. In some embodiments, the protein is an enzyme, a regulatory protein, or a structural protein, e.g., one that can substitute for a missing or defective protein in a subject. The protein may be one that is secreted from a cell or one that is not secreted from a cell. In some embodiments, the nucleic acid is a functional nucleic acid, e.g., an antisense nucleic acid, an inhibitory RNA, or an aptamer.

Any nucleic acid sequence(s) of interest may be delivered in the expression vectors of the present invention. Nucleic acids of interest include nucleic acids encoding polypeptides, including therapeutic (e.g., for medical or veterinary uses), immunogenic (e.g., for vaccines), or diagnostic polypeptides.

Therapeutic polypeptides include, but are not limited to, cystic fibrosis transmembrane regulator protein (CFTR), dystrophin (including mini- and micro-dystrophins (see, e.g, Vincent et al., (1993) Nature Genetics 5:130; U.S. Patent Publication No. 2003/017131; International publication WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, α₁-antitrypsin, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-α and -β, and the like), lysosomal acid α-glucosidase, α-galactosidase A, receptors (e.g., the tumor necrosis growth factorα soluble receptor), S100A1, parvalbumin, adenylyl cyclase type 6, a molecule that effects G-protein coupled receptor kinase type 2 knockdown such as a truncated constitutively active bARKct, anti-inflammatory factors such as IRAP, anti-myostatin proteins, aspartoacylase, and monoclonal antibodies (including single chain monoclonal antibodies; an exemplary Mab is the Herceptin® Mab). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. Parvovirus vectors can also be used to deliver monoclonal antibodies and antibody fragments, for example, an antibody or antibody fragment directed against myostatin (see, e.g., Fang et al., Nature Biotechnol. 23:584-590 (2005)).

In particular embodiments, the therapeutic protein is heparan alpha-glucosaminide N-acetyltransferase (HGSNAT) or survival motor neuron 1 (SMN1).

Nucleic acid sequences encoding polypeptides include those encoding reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene.

Alternatively, in particular embodiments of this invention, the nucleic acid may encode a functional nucleic acid, i.e., nucleic acid that functions without getting translated into a protein, e.g., an antisense nucleic acid, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), RNAi to a sarcoglycan [e.g., α, β, γ], RNAi against myostatin, myostatin propeptide, follistatin, or activin type II soluble receptor, RNAi against anti-inflammatory polypeptides such as the Ikappa B dominant mutant, and RNAi directed against pathogenic organisms and viruses (e.g., hepatitis B virus, human immunodeficiency virus, CMV, herpes simplex virus, human papilloma virus, etc.).

Alternatively, in particular embodiments of this invention, the nucleic acid may encode protein phosphatase inhibitor I (I-1), serca2a, zinc finger proteins that regulate the phospholamban gene, Barkct, β2-adrenergic receptor, β2-adrenergic receptor kinase (BARK), phosphoinositide-3 kinase (PI3 kinase), a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, enos, inos, or bone morphogenic proteins (including BNP 2, 7, etc., RANKL and/or VEGF).

The expression vectors may also comprise a nucleic acid that shares homology with and recombines with a locus on a host chromosome. This approach can be utilized, for example, to correct a genetic defect in the host cell.

The present invention also provides expression vectors that express an immunogenic polypeptide, e.g., for vaccination. The nucleic acid may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura et al., (1994) Proc. Nat. Acad. Sci USA 91:8507; U.S. Pat. No. 5,916,563 to Young et al., U.S. U.S. Pat. No. 5,905,040 to Mazzara et al., U.S. Pat. No. 5,882,652, 5,863,541 to Samulski et al.). The antigen may be presented in the parvovirus capsid. Alternatively, the antigen may be expressed from a nucleic acid introduced into a recombinant vector genome. Any immunogen of interest as described herein and/or as is known in the art can be provided by the expression vectors.

An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogenic polypeptide can also be an arenavirus immunogen (e.g., Lassa fever virus immunogen, such as the Lassa fever virus nucleocapsid protein and the Lassa fever envelope glycoprotein), a poxvirus immunogen (e.g., a vaccinia virus immunogen, such as the vaccinia L1 or L8 gene products), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpes immunogen (e.g., CMV, EBV, HSV immunogens) a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) (′ancer Res. 54:3124), MART-1, gp100 MAGE-1, MAGE-2, MAGE-3, CEA, TRP-1, TRP-2, P-15, tyrosinase (Brichard et al., (1993) J. Exp. Med. 178:489); HER-2/neu gene product (U.S. Pat. No. 4,968,603), CA 125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

It will be understood by those skilled in the art that the nucleic acid(s) of interest can be operably associated with appropriate control sequences. For example, the heterologous nucleic acid can be operably associated with expression control elements, such as transcription/translation control signals, origins of replication, polyadenylation signals (e.g., the soluble neuropilin-1 (sNRP-1) Poly A signal), internal ribosome entry sites (IRES), promoters, and/or enhancers, and the like.

Those skilled in the art will appreciate that a variety of promoter/enhancer elements can be used depending on the level and tissue-specific expression desired. The promoter/enhancer can be constitutive (e.g., the cytomegalovirus promoter or miniature cytomegalovirus promoter) or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

### Methods of Use

A further aspect of the invention relates to methods of delivering a nucleic acid of interest to bystander cells in a subject, comprising administering to the subject an effective amount of the expression vector, virus particle, or pharmaceutical composition of the invention, thereby forming extracellular vesicles comprising the nucleic acid of interest and delivering the nucleic acid of interest to bystander cells. The method of the invention advantageously may result in a therapeutically significant number of cells expressing the nucleic acid of interest without having to transduce each cell with the expression vector or viral particle.

An additional aspect of the invention relates to methods of treating a disorder treatable by expression of a nucleic acid of interest in a subject in need thereof, comprising administering to the subject a therapeutically effective amount of the expression vector, virus particle, or pharmaceutical composition of the invention, thereby treating the disorder.

In some embodiments, the therapeutic protein is HGSNAT and the disorder is mucopolysaccharidosis IIIC. In some embodiments, the therapeutic protein is SMN1 and the disorder is spinal muscle atrophy. In some embodiments, the disorder is fragile X syndrome, Niemann-Pick disease type C, neuronal ceroid lipofuscinoses, Charcot-Marie-Tooth Disease, Friedreich's Ataxia, or a neuromuscular disorder.

As used herein, “transduction” of a cell by an expression vector, e.g., a virus vector (e.g., an AAV vector) means entry of the vector into the cell and transfer of genetic material into the cell by the incorporation of nucleic acid into the virus vector and subsequent transfer into the cell via the virus vector.

Unless indicated otherwise, “efficient transduction” or “efficient tropism,” or similar terms, can be determined by reference to a suitable positive or negative control (e.g., at least about 50%, 60%, 70%, 80%, 85%, 90%, 95% or more of the transduction or tropism, respectively, of a positive control or at least about 110%, 120%, 150%, 200%, 300%, 500%, 1000% or more of the transduction or tropism, respectively, of a negative control). Similarly, it can be determined if a virus “does not efficiently transduce” or “does not have efficient tropism” for a target tissue, or similar terms, by reference to a suitable control. In particular embodiments, the virus vector does not efficiently transduce (i.e., does not have efficient tropism for) tissues outside the CNS or tissues other than muscle, e.g., liver, kidney, gonads and/or germ cells. In particular embodiments, undesirable transduction of tissue(s) (e.g., liver) is 20% or less, 10% or less, 5% or less, 1% or less, 0.1% or less of the level of transduction of the desired target tissue(s) (e.g., CNS cells or muscle cells).

Those skilled in the art will appreciate that transcription of a heterologous nucleic acid sequence from a viral genome may not be initiated in the absence of trans-acting factors, e.g., for an inducible promoter or otherwise regulated nucleic acid sequence. In the case of a rAAV genome, gene expression from the viral genome may be from a stably integrated provirus and/or from a non-integrated episome, as well as any other form which the virus nucleic acid may take within the cell.

A “bystander cell” is a cell in a subject that has not been transduced by an expression vector after administration of the expression vector to the subject. The ability of the expression vectors of the invention to incorporate mRNA produced from the nucleic acid of interest into EVs, have the EVs deliver the mRNA to bystander cells, and have functional protein expressed in the bystander cells is advantageous in gene therapy methods.

The cell(s) into which the expression vector is introduced can be of any type, including but not limited to neural cells (including cells of the peripheral and central nervous systems, in particular, brain cells such as neurons and oligodendrocytes), lung cells, cells of the eye (including retinal cells, retinal pigment epithelium, and corneal cells), blood vessel cells (e.g., endothelial cells, intimal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, kidney cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above. Furthermore, the cells may be dividing or non-dividing.

Embodiments of the invention may be performed in vitro or in vivo. One aspect of the present invention is a method of transferring an expression vector to a cell in vitro, e.g., for research purposes or as part of an ex vivo method. The expression vector may be introduced into the cells at the appropriate amount, e.g., multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, more preferably at least about 10⁵ infectious units are introduced to the cell.

In particular embodiments, the cells have been removed from a subject, the expression vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the expression vector can be introduced into cells from a donor subject, into cultured cells, or into cells from any other suitable source, and the cells are administered to a subject in need thereof (i.e., a “recipient” subject).

Suitable cells for ex vivo gene delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the expression vector are administered to the subject in a treatment effective or prevention effective amount in combination with a pharmaceutical carrier.

The expression vector are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The expression vector can also be used to produce a polypeptide of interest or functional RNA in a subject (e.g., using the subject as a bioreactor to produce the polypeptide or to observe the effects of the functional nucleic acid on the subject, for example, in connection with screening methods). The expression vector may also be employed to provide a functional nucleic acid to a cell in vitro or in vivo. Expression of the functional nucleic acid in the cell, for example, can diminish expression of a particular target protein by the cell. Accordingly, functional nucleic acid can be administered to decrease expression of a particular protein in a subject in need thereof.

Expression vector also find use in diagnostic and screening methods, whereby a nucleic acid of interest is transiently or stably expressed in a transgenic animal model.

The expression vector can also be used for various non-therapeutic purposes, including but not limited to use in protocols to assess gene targeting, clearance, transcription, translation, etc., as would be apparent to one skilled in the art. The expression vector can also be used for the purpose of evaluating safety (spread, toxicity, immunogenicity, etc.). Such data, for example, are considered by the United States Food and Drug Administration as part of the regulatory approval process prior to evaluation of clinical efficacy.

### Pharmaceutical Formulations, Subjects, and Modes of Administration

Provided according to embodiments of the invention are compositions that include an expression vector or virus particle of the invention. Also provided herein are pharmaceutical compositions comprising an expression vector or virus particle of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form. The present invention also provides a complex between the expression vector or virus particle in a pharmaceutically acceptable carrier and, optionally, other medicinal agents, pharmaceutical agents, stabilizing agents, buffers, carriers, adjuvants, diluents, etc. By “pharmaceutically acceptable” it is meant a material that is not toxic or otherwise undesirable, i.e., the material may be administered to a subject without causing any undesirable biological effects.

A further aspect of the invention is a method of administering the expression vector or virus particle of the invention to subjects. Administration of the expression vector or virus particle of the invention to a human subject or an animal in need thereof can be by any means known in the art. Optionally, the expression vector or virus particle of the invention is delivered in a treatment effective or prevention effective dose in a pharmaceutically acceptable carrier.

Injectables can be prepared in conventional forms, either as liquid solutions or suspensions, solid forms suitable for solution or suspension in liquid prior to injection, or as emulsions. Alternatively, one may administer the expression vector or virus particle of the invention in a local rather than systemic manner, for example, in a depot or sustained-release formulation. Further, the expression vector or virus particle of the invention can be delivered adhered to a surgically implantable matrix (e.g., as described in U.S. Patent Publication No. 2004-0013645). The expression vector or virus particle of the invention disclosed herein can be administered to the lungs of a subject by any suitable means, optionally by administering an aerosol suspension of respirable particles comprised of the expression vector or virus particle of the invention, which the subject inhales. The respirable particles can be liquid or solid. Aerosols of liquid particles comprising the expression vector or virus particle of the invention may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the expression vector or virus particle of the invention may likewise be produced with any solid particulate medicament aerosol generator, by techniques known in the pharmaceutical art.

In certain embodiments, the expression vector or virus particle of the invention are administered to a subject in need thereof as early as possible in the life of the subject, e.g., as soon as the subject is diagnosed with a disease or disorder. In some embodiments, the method are carried out on a newborn subject, e.g., after newborn screening has identified a disease or disorder. In some embodiments, methods are carried out on a subject prior to the age of 10 years, e.g., prior to 1, 2, 3, 4, 5, 6, 7, 8, 9, or 10 years of age. In some embodiments, the methods are carried out on juvenile or adult subjects after the age of 10 years. In some embodiments, the methods are carried out on a fetus in utero, e.g., after prenatal screening has identified a disease or disorder. In some embodiments, the methods are carried out on a subject as soon as the subject develops symptoms associated with a disease or disorder. In some embodiments, the methods are carried out on a subject before the subject develops symptoms associated with a disease or disorder, e.g., a subject that is suspected or diagnosed as having a disease or disorder but has not started to exhibit symptoms.

The expression vector or virus particle of the invention may be administered to a subject by any route of administration found to be effective to provide expression of the nucleic acid of interest. The most suitable route will depend on the subject being treated and the disorder or condition being treated. In some embodiments, the expression vector or virus particle of the invention is administered to the subject by a route selected from oral, rectal, transmucosal, intranasal, inhalation (e.g., via an aerosol), buccal (e.g., sublingual), vaginal, intrathecal, intraocular, intravitreal, intracochlear, transdermal, intraendothelial, in utero (or in ovo), parenteral (e.g., intravenous, subcutaneous, intradermal, intracranial, intrathecal, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

In particular embodiments, more than one administration (e.g., two, three, four or more administrations) may be employed to achieve the desired level of gene expression over a period of various intervals, e.g., daily, weekly, monthly, yearly, etc.

The expression vector or virus particle of the invention can be administered to tissues of the CNS (e.g., brain, eye) and may advantageously result in broader distribution of the expression vector or virus particle than would be observed in the absence of the present invention.

Administration can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye.

Administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoideohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

The expression vector or virus particle of the invention can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105:3458-3464), intrathecal administration, and/or direct intramuscular injection. In particular embodiments, the expression vector or virus particle are administered to a limb (arm and/or leg) of a subject (e.g., a subject with muscular dystrophy such as DMD) by limb perfusion, optionally isolated limb perfusion (e.g., by intravenous or intraarticular administration. In embodiments of the invention, the expression vector or virus particle can advantageously be administered without employing “hydrodynamic” techniques. Tissue delivery (e.g., to muscle) of prior art vectors is often enhanced by hydrodynamic techniques (e.g., intravenous/intravenous administration in a large volume), which increase pressure in the vasculature and facilitate the ability of the agent to cross the endothelial cell barrier. In particular embodiments, the expression vector or virus particle can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome.

Administration to cardiac muscle includes administration to the left atrium, right atrium, left ventricle, right ventricle and/or septum. The expression vector or virus particle of the invention can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration to diaphragm muscle can be by any suitable method including intravenous administration, intra-arterial administration, and/or intra-peritoneal administration.

Administration to smooth muscle can be by any suitable method including intravenous administration, intra-arterial administration, and/or intra-peritoneal administration. In one embodiment, administration can be to endothelial cells present in, near, and/or on smooth muscle.

Delivery to a target tissue can also be achieved by delivering a depot comprising the expression vector or virus particle of the invention. In representative embodiments, a depot comprising the expression vector or virus particle is implanted into skeletal, smooth, cardiac and/or diaphragm muscle tissue or the tissue can be contacted with a film or other matrix comprising the expression vector or virus particle. Such implantable matrices or substrates are described in U.S. Pat. No. 7,201,898.

Administration can also be to a tumor (e.g., in or near a tumor or a lymph node). The most suitable route in any given case will depend on the nature and severity of the condition being treated and/or prevented and on the nature of the particular vector that is being used.

The expression vector or virus particle of the invention may be delivered or targeted to any tissue or organ in the subject. In some embodiments, the expression vector or virus particle are administered to, e.g., a skeletal muscle, a smooth muscle, the heart, the diaphragm, the airway epithelium, the liver, the kidney, the spleen, the pancreas, the skin, the lung, the brain, the spinal cord, the ear, and the eye. In some embodiments, the expression vector or virus particle is administered to a diseased tissue or organ, e.g., a tumor.

In general, the expression vector or virus particle of the invention can be employed to deliver a nucleic acid encoding a polypeptide or functional nucleic acid to treat and/or prevent any disease state for which it is beneficial to deliver a therapeutic polypeptide or functional nucleic acid. Illustrative disease states include, but are not limited to: fragile X syndrome, neuronal ceroid lipofuscinoses, Charcot-Marie-Tooth Disease, Friedreich's Ataxia, cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the Ikappa B dominant mutant, sarcospan, utrophin, mini-utrophin, RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic defects, congenital emphysema (α1-antitrypsin), Lesch-Nyhan Syndrome (hypoxanthine guanine phosphoribosyl transferase), Niemann-Pick disease (sphingomyelinase), Tays Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1), serca2a, zinc finger proteins that regulate the phospholamban gene, Barkct, β2-adrenergic receptor, β2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), neuromuscular disorders, autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

In particular embodiments, expression vector or virus particle of the invention is administered to skeletal muscle, diaphragm muscle and/or cardiac muscle (e.g., to treat and/or prevent muscular dystrophy or heart disease [for example, PAD or congestive heart failure]).

Gene transfer has substantial potential use for understanding and providing therapy for disease states. There are a number of inherited diseases in which defective genes are known and have been cloned (i.e., disorders treatable by expression of a nucleic acid of interest). In general, the above disease states fall into two classes: deficiency states, usually of proteins/enzymes, which are generally inherited in a recessive manner, and unbalanced states, which may involve regulatory or structural proteins, and which are typically inherited in a recessive manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, expression vectors permit the treatment and/or prevention of genetic diseases.

As a further aspect, the expression vectors of the present invention may be used to produce an immune response in a subject. According to this embodiment, expression vectors comprising a nucleic acid sequence encoding an immunogenic polypeptide can be administered to a subject, and an active immune response is mounted by the subject against the immunogenic polypeptide. Immunogenic polypeptides are as described hereinabove. In some embodiments, a protective immune response is elicited.

Alternatively, the expression vectors may be administered to a cell ex vivo and the altered cell is administered to the subject. The expression vectors comprising the nucleic acid is introduced into the cell, and the cell is administered to the subject, where the nucleic acid encoding the immunogen can be expressed and induce an immune response in the subject against the immunogen. In particular embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof. In particular embodiments, the expression vector or cell comprising the nucleic acid can be administered in an immunogenically effective amount, as described below.

The expression vectors can also be administered for cancer immunotherapy by administration of expression vectors expressing one or more cancer cell antigens (or an immunologically similar molecule) or any other immunogen that produces an immune response against a cancer cell. To illustrate, an immune response can be produced against a cancer cell antigen in a subject by administering expression vectors comprising a nucleic acid encoding the cancer cell antigen, for example to treat a patient with cancer and/or to prevent cancer from developing in the subject. The expression vectors may be administered to a subject in vivo or by using ex vivo methods, as described herein. Alternatively, the cancer antigen can be expressed as part of the expression vectors.

As another alternative, any other therapeutic nucleic acid (e.g., RNAi) or polypeptide (e.g., cytokine) known in the art can be administered to treat and/or prevent cancer.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In representative embodiments, the invention provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In particular embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

In particular embodiments, cells may be removed from a subject with cancer and contacted with the expression vector or virus particle of the invention. The modified cell is then administered to the subject, whereby an immune response against the cancer cell antigen is elicited. This method can be advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, β-interferon, γ-interferon, ω-interferon, τ-interferon, interleukin-1α, interleukin-1β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-«, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the expression vector or virus particle.

Cytokines may be administered by any method known in the art. Exogenous cytokines may be administered to the subject, or alternatively, a nucleic acid encoding a cytokine may be delivered to the subject using a suitable vector, and the cytokine produced in vivo.

The methods of the present invention find use in both veterinary and medical applications. Suitable subjects include avians, reptiles, amphibians, fish, and mammals. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disorder including those described herein or that would benefit from the delivery of a polynucleotide including those described herein. As a further option, the subject can be a laboratory animal and/or an animal model of disease. Preferably, the subject is a human.

In some embodiments, the expression vector or virus particle of the invention is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above). The degree of protection conferred need not be complete or permanent, as long as the benefits of administering the immunogenic polypeptide outweigh any disadvantages thereof.

The expression vector or virus particle of the invention can further be administered to elicit an immunogenic response (e.g., as a vaccine). Typically, immunogenic compositions of the present invention comprise an immunogenically effective amount of the expression vector or virus particle in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the expression vector (e.g., viral vector) to be administered to a subject depend upon the mode of administration, the disease or condition to be treated and/or prevented, the individual subject's condition, the particular expression vector, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸ transducing units, optionally about 10⁸-10¹⁵ transducing units. In some embodiments, the dosage of the expression vector is less than what would be needed to achieve a therapeutic effect with an expression vector that does not comprise an extracellular vesicle-targeting zip code sequence.

In a representative embodiment, the invention provides a method of treating and/or preventing muscular dystrophy in a subject in need thereof, the method comprising: administering a treatment or prevention effective amount of expression vector or virus particle of the invention to a mammalian subject, wherein the expression vector or virus particle comprises a nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the expression vector or virus particle can be administered to skeletal, diaphragm and/or cardiac muscle as described elsewhere herein.

Alternatively, the invention can be practiced to deliver a nucleic acid to skeletal, cardiac or diaphragm muscle, which is used as a platform for production of a polypeptide (e.g., an enzyme) or functional nuclei acid (e.g., functional RNA, e.g., RNAi, microRNA, antisense RNA) that normally circulates in the blood or for systemic delivery to other tissues to treat and/or prevent a disorder (e.g., a metabolic disorder, such as diabetes (e.g., insulin), hemophilia (e.g., Factor IX or Factor VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid α-glucosidase] or Fabry disease [α-galactosidase A]) or a glycogen storage disorder (such as Pompe disease [lysosomal acid α glucosidase]). Other suitable proteins for treating and/or preventing metabolic disorders are described above. The use of muscle as a platform to express a nucleic acid of interest is described in U.S. Patent Publication No. 2002/0192189.

Thus, as one aspect, the invention further encompasses a method of treating and/or preventing a metabolic disorder in a subject in need thereof, the method comprising: administering a treatment or prevention effective amount of expression vector or virus particle of the invention to a subject (e.g., to skeletal muscle of a subject), wherein the expression vector or virus particle comprises a nucleic acid encoding a polypeptide, wherein the metabolic disorder is a result of a deficiency and/or defect in the polypeptide. Illustrative metabolic disorders and nucleic acids encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art). Without being limited by any particular theory of the invention, according to this embodiment, administration to the skeletal muscle can result in secretion of the polypeptide into the systemic circulation and delivery to target tissue(s). Methods of delivering the expression vector or virus particle of the invention to skeletal muscle are described in more detail herein.

The invention can also be practiced to produce antisense RNA, RNAi or other functional RNA (e.g., a ribozyme) for systemic delivery.

The invention also provides a method of treating and/or preventing congenital heart failure or PAD in a subject in need thereof, the method comprising administering a treatment or prevention effective amount of the expression vector or virus particle of the invention to a mammalian subject, wherein the expression vector or virus particle comprises a nucleic acid encoding, for example, a sarcoplasmic endoreticulum Ca²⁺-ATPase (SERCA2a), an angiogenic factor, phosphatase inhibitor I (I-1), RNAi against phospholamban; a phospholamban inhibitory or dominant-negative molecule such as phospholamban S16E, a zinc finger protein that regulates the phospholamban gene, β2-adrenergic receptor, β2-adrenergic receptor kinase (BARK), PI3 kinase, calsarcan, a β-adrenergic receptor kinase inhibitor (BARKct), inhibitor 1 of protein phosphatase 1, S100A1, parvalbumin, adenylyl cyclase type 6, a molecule that effects G-protein coupled receptor kinase type 2 knockdown such as a truncated constitutively active bARKct, Pim-1, PGC-1α, SOD-1, SOD-2, EC-SOD, kallikrein, HIF, thymosin-β4, mir-1, mir-133, mir-206 and/or mir-208.

In particular embodiments, the expression vector or virus particle of the invention may be administered to treat diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, spinal muscle atrophy, progressive muscular atrophy, mucopolysaccharidosis, fragile X syndrome, neuronal ceroid lipofuscinoses, Charcot-Marie-Tooth Disease, Friedreich's Ataxia, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulemia) and cancers and tumors (e.g., pituitary tumors) of the CNS.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The expression vector or virus particle of the invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a expression vector or virus particle of the invention.

Retinitis pigmentosa, by comparison, is characterized by retinal degeneration. In representative embodiments, retinitis pigmentosa can be treated by intraocular (e.g., vitreal administration) of the expression vector or virus particle of the invention encoding one or more neurotrophic factors.

Age-related macular degeneration involves both angiogenesis and retinal degeneration. This disorder can be treated by administering the expression vector or virus particle of the invention encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the expression vector or virus particle of the invention. Such agents include N-methyl-D-aspartate (NMDA) antagonists, cytokines, and neurotrophic factors, delivered intraocularly, optionally intravitreally.

other embodiments, the present invention may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

In one representative embodiment, somatostatin (or an active fragment thereof) is administered to the brain using the expression vector or virus particle of the invention to treat a pituitary tumor. According to this embodiment, the expression vector or virus particle encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins as are known in the art.

In particular embodiments, the expression vector or virus particle of the invention can comprise a secretory signal as described in U.S. Pat. No. 7,071,172.

In representative embodiments of the invention, the expression vector or virus particle of the invention is administered to the CNS (e.g., to the brain or to the eye). The expression vector or virus particle may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The expression vector or virus particle may also be administered to different regions of the eye such as the retina, cornea and/or optic nerve.

The expression vector or virus particle of the invention may be delivered into the cerebrospinal fluid (e.g., by lumbar puncture) for more disperse administration of the expression vector or virus particle. The expression vector or virus particle may further be administered intravascularly to the CNS in situations in which the blood-brain barrier has been perturbed (e.g., brain tumor or cerebral infarct).

The expression vector or virus particle of the invention can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In particular embodiments, the expression vector or virus particle of the invention is administered in a liquid formulation by direct injection (e.g., stereotactic injection) to the desired region or compartment in the CNS. In other embodiments, the expression vector or virus particle may be provided by topical application to the desired region or by intra-nasal administration of an aerosol formulation. Administration to the eye, may be by topical application of liquid droplets. As a further alternative, the expression vector or virus particle may be administered as a solid, slow-release formulation (see, e.g., U.S. Pat. No. 7,201,898).

In yet additional embodiments, the expression vector or virus particle of the invention can used for retrograde transport to treat and/or prevent diseases and disorders involving motor neurons (e.g., amyotrophic lateral sclerosis (ALS); spinal muscular atrophy (SMA), etc.). For example, the expression vector or virus particle can be delivered to muscle tissue from which it can migrate into neurons.

Having described the present invention, the same will be explained in greater detail in the following examples, which are included herein for illustration purposes only, and which are not intended to be limiting to the invention.

### EXAMPLES

### Example 1

### MPS IIIC Studies

In preliminary studies, the disclosed rAAV product was constructed by incorporation of an EV-mRNA packaging signal to a rAAV-hHGSNAT gene replacement vector to facilitate the by-stander effect. The vector construct was tested using an in vitro model of MPS IIIC, to assess its therapeutic potential.

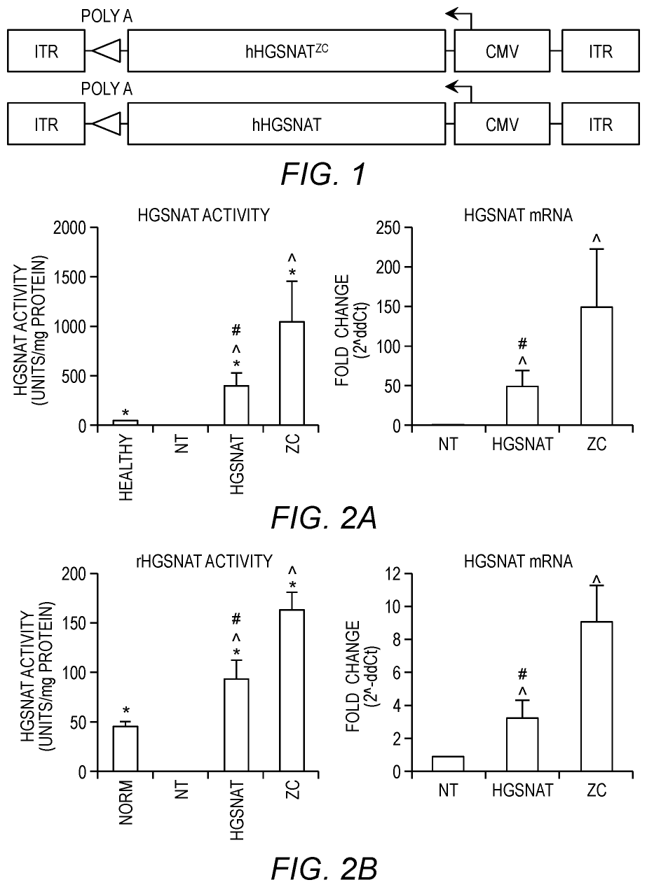

Construction of rAAV-hHGSNAT vectors: A rAAV vector plasmid, ptr-CMV-hHGSNAT^{zc} (SEQ ID NO:2), was constructed to express hHGSNAT, containing a 25 nt EV zip-code (ZC) signal sequence for packaging hHGSNAT-mRNA into EVs. A second AAV vector plasmid, ptr-CMV-hHGSNAT (SEQ ID NO:3), without the EV signal was constructed as a control. These plasmids were used to produce rAAV-CMV-hHGSNAT^{zc} and rAAV-CMV-hHGSNAT viral vectors. The rAAV-hHGSNAT viral vector genome (vg) contains only minimal elements required for transgene expression (FIG. 1).

rAAV-mediated expression of functional rHGSNAT and EV-hHGSNAT-mRNA packaging: To determine the function of these vector constructs, they were first tested in vitro in Hela cells by transfection with the vector plasmids, ptr-CMV-hHGSNAT or ptr-CMV-hHGSNAT^{zc}. At 48 h post transfection, cell lysates were assayed for rHGSNAT expression by HGSNAT activity assay and qRT-PCR. The results showed significantly increased HGSNAT activity and mRNA in the transfected cells, compared to non-transfected Hela cells These data indicate both vectors mediated effective expression of functional rHGSNAT.

To further assess the therapeutic potential of the rAAV-hHGSNAT vectors, they were tested in human MPS IIIC skin fibroblast (GM05157) cultures by transfection with the vector plasmids and by infection with rAAV2 viral vectors. Healthy human skin fibroblasts (GM00969) and untreated GM05157 cells were used as controls. GM05157 cells in 10 cm plates were transfected with plasmid ptr-CMV-hHGSNAT or ptr-CMV-hHGSNAT^{zc} (7 μg/plate), or infected with rAAV2-CMV-hHGSNAT or rAAV2-CMV-hHGSNAT^{zc} viral vectors (1×10³ vg/cell). At 48 h post transduction, cell lysates were assayed in duplicate for rHGSNAT expression by HGSNAT activity assay and qRT-PCR (FIGS. 2A-2B). Media samples were processed for EV extraction, transfer, and analysis. As a result, both AAV vector constructs mediated effective rHGSNAT expression by transfection (FIG. 2A) and rAAV2 infection (FIG. 2B). While the mechanisms are unclear, inclusion of the 25 nt EV ZC signal sequence was shown to enhance rHGSNAT expression at both the protein and mRNA levels. Transfections appear to induce higher levels of rHGSNAT expression than infection, likely because more vector plasmids could enter the cells (via bulk transport) than rAAV2 viral vectors (via receptor-mediated uptake). Notably, the experiments of transfection and vector infection were conducted in duplicate and repeated ≥2 times.

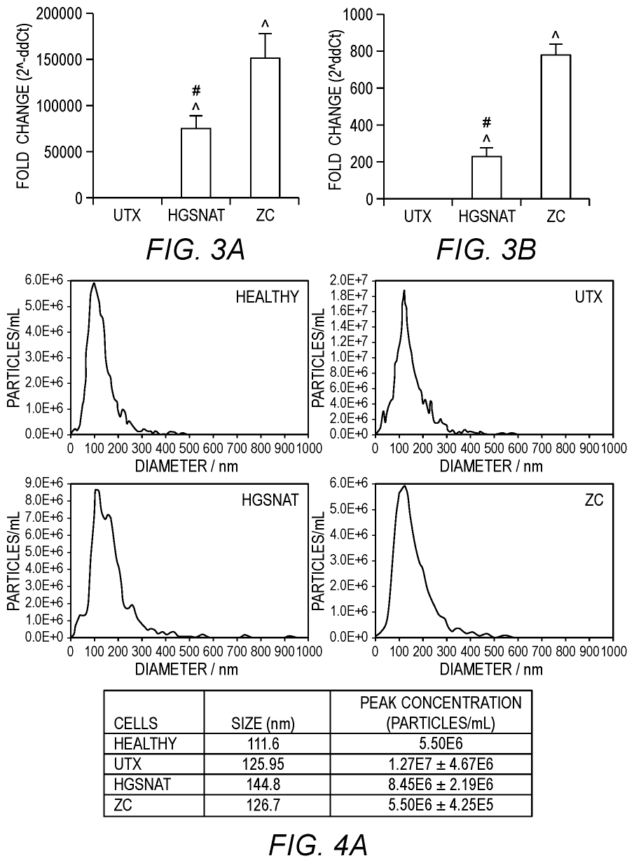

To determine whether including the 25 nt EV ZC signal sequence in the vector mediates EV-HGSNAT-mRNA packaging, media samples from the transduced and control cells were processed to isolate EVs by ultracentrifugation. The isolated EVs were processed to extract RNA and EV RNA samples were assayed for hHGSNAT mRNA by qRT-PCR. The results showed that both AAV-hHGSNAT^{zc} and AAV-hHGSNAT vectors mediated the secretion of hHGSNAT-mRNA-containing EVs from cells transduced by transfection (FIG. 3A) and infection (FIG. 3B). However, the EV ZC tag was shown to mediate much higher levels of EV-hHGSNAT-mRNA packaging. While the mechanism is unclear, it is possible that the transduction of AAV-hHGSNAT vectors without EV ZC may also induce EV-hHGSNAT-mRNA packaging as a biophysiological response. Similarly, plasmid transfections appear to induce much higher levels of EV rHGSNAT mRNA than rAAV2 infection, congruent with the previously observed higher level intracellular expression. Together, these observations support the hypothesis that the EV ZC signal can mediate effective EV-hHGSNAT-mRNA cargo by AAV gene delivery.

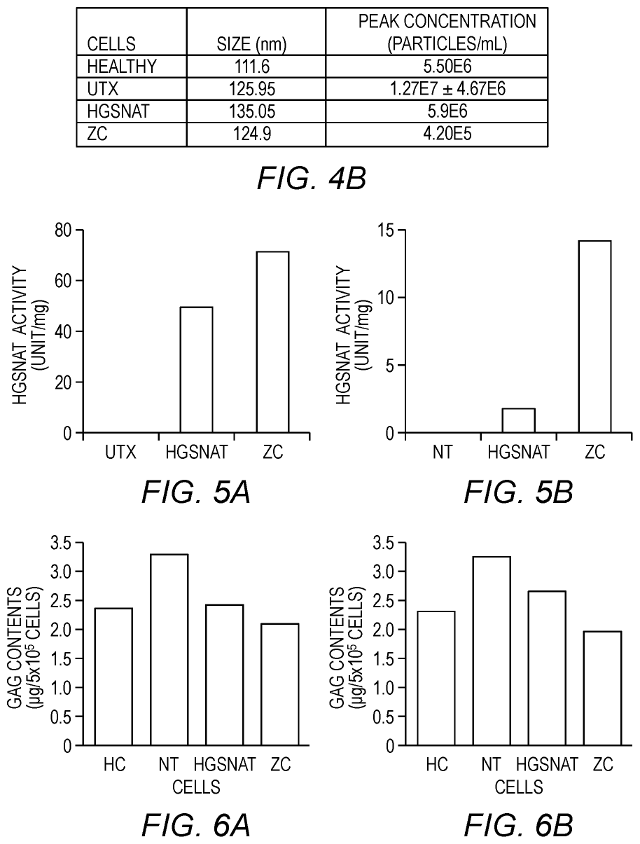

Characteristics of AAV-mediated EVs in human MPS IIIC skin fibroblasts: The EV samples were characterized using NanoSight NS500 at UNC Nanomedicines Characterization Core Facility, to visualize the EVs, and determine the quantity and size distribution of EVs. The isolated EVs were shown to be medium sized (111.6-144.8 nm) (FIGS. 4A-4B). Interestingly, while the same number of cells were used among different experimental groups, about 2-fold more EVs were detected from non-treated MPS IIIC cells than from healthy cells or MPS IIIC cells treated by transfection (FIG. 4A) or infection (FIG. 4B) with both of the AAV-hHGSNAT vector constructs with or without the EV ZC signal. Notably, an increase in EVs has been linked to many pathological conditions, such as autoimmune diseases and cancer. Therefore, the increase in EVs from MPS IIIC cells may be due to the disease pathophysiology. Importantly, the EV reduction following AAV-hHGSNAT transduction suggests the correction of the pathological condition, supporting the therapeutic potential of AAV-hHGSNAT gene therapy.

EV-mRNA transfer leading to the expression of functional rHGSNAT protein in recipient MPS IIIC cells: To determine whether AAV-hHGSNAT-mediated EVs can facilitate bystander effects, GM05157 cells were incubated in duplicate with EVs that had been isolated from the media of MPS IIIC cells treated with AAV-hHGSNAT vectors by plasmid transfection (FIG. 5A) or AAV2 infection (FIG. 5B). At 48 h of incubation, the recipient cell lysates were assayed for HGSNAT enzyme activity. The results showed that EVs from both transfected or infected cells resulted in HGSNAT activity in MPS IIIC cells, indicating that HGSNAT-mRNA was transferred to the cells by EVs and translated into functional HGSNAT protein. EVs from cells transduced with AAV-hHGSNAT^{zc} led to higher mRNA transfer (FIGS. 2A-2B and 3A-3B). Again, transfection appears to induce more EV-mediated rHGSNAT protein expression (FIGS. 5A-5B), compared to rAAV2 infection. These data demonstrate that tagging the 25 nt EV ZC signal to rAAV-hHGSNAT vector facilitates potentially efficacious bystander effects in untransduced MPS IIIC cells, supporting the therapeutic potential of this approach for treating neurogenetic diseases involving non-secreted proteins.

Clearance of GAG storage in vitro in MPS IIIC cells by rAAV-hHGSNAT gene transfer and EV-hHGSNAT-mRNA cargo: To further assess the function of the rAAV9-mediated rHGSNAT, human MPS IIIC skin fibroblasts (GM05157) were transfected in duplicate with plasmid ptr-CMV-hHGSNAT or ptr-CMV-hHGSNAT^{zc} (7 μg/plate), and the media from transfected cells were used to culture non-treated GM05157 cells. At 48 h post transfection or media incubation, cell lysates were assayed in duplicate for GAG content. The results showed the reduction of GAG content to normal levels in cells transduced with both vector constructs (FIG. 6A), further demonstrating that the AAV-mediated rHGSNAT are functional and led to the clearance of GAG storage in MPS IIIC cells. Importantly, incubation with media of hHGSNAT^{zc}-transduced cells resulted in complete correction of GAG storage in MPS IIIC cells (FIG. 6B), indicating that ZC-mediated EV cargo led to the translation of HGSNAT-mRNA to functional rHGSNAT protein in non-transduced MPS IIIC cells. Notably, incubation with media from hHGSNAT-transduced cells led to partial GAG reduction in MPS IIIC cells (FIG. 6B), suggesting that transduction by ptr-hHGSNAT vector may also result in a low level of EV-HGSNAT-mRNA packaging, though the mechanisms are unclear. Correlating with data presented in FIGS. 2A-2B, 3A-3B, and 5A-5B, these results further demonstrate the therapeutic potential of the bystander effects facilitated by the AAV-mediated EV-hHGSNAT-mRNA cargo for treating MPS IIIC. The EV-facilitated cross-correction allows significant therapeutic benefit from a small number of transduced cells within a tissue, as long as they are well distributed.

In summary, an rAAV-hHGSNAT vector containing a 25 nt EV ZC for mRNA cargo was developed. The studies showed that the rAAV-hHGSNAT^{zc} mediates efficient rHGSNAT expression and EV-HGSNAT-mRNA packaging. Importantly, these EVs can transfer HGSNAT-mRNA to and mediate the expression of rHGSNAT protein in untreated MPS IIIC cells. These data demonstrate that rAAV-hHGSNAT vector containing the 25 nt EV ZC can mediate not only efficient rHGSNAT expression, but also EV-HGSNAT-mRNA cargo. The EVs can transfer their rHGSNAT-mRNA contents to the recipient cells, where the rHGSNAT-mRNA can be translated to functional rHGSNAT protein leading to the clearance of GAG storage. The demonstrated EV-facilitated bystander effect offers great potential for treating MPS IIIC using rAAV gene replacement therapy. This bystander effect may allow optimal therapeutic benefits via cross-correction, without having to transduce every single cell, because the transduced cells serve as factories producing rHGSNAT-mRNA-containing EVs that can enter the neighboring cells, where the rHGSNAT-mRNAs translate to functional proteins.

AAV normally packages a single-stranded (ss) DNA genome that must be converted to double-strand (ds) DNA after infection. Constructs less than half or less of the normal WT AAV genome size can be packaged as dimeric inverted repeat DNA molecules. These self-complementary (sc) genomes fold into ds DNA as soon as they are released from the capsid, bypassing the requirement for DNA synthesis by the host cells to convert the single-stranded (ss) vg into active double-stranded DNA and providing faster and more efficient transduction.

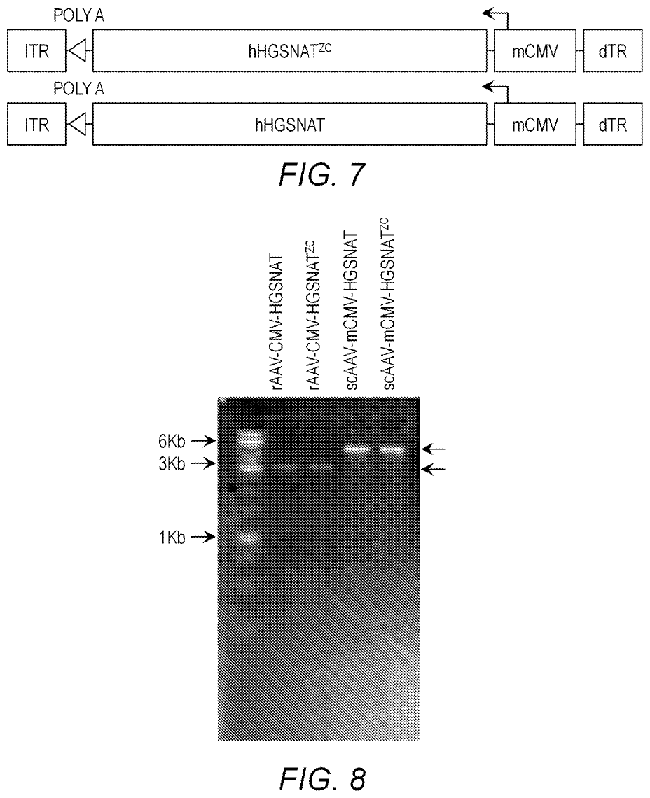

A set of two self-complementary AAV (sc) vector expressing rhHGSNAT were constructed for added transduction efficiency. The scAAV-mCMV-hHGSNAT^{zc} vector (SEQ ID NO: 4) was constructed that contains the 25 nt ZC sequence linked to hHGSNAT cDNA and the scAAV-mCMV-hHGSNAT vector (SEQ ID NO:5) without the ZC signal. To accommodate the vector genome size limit for AAV vector packaging (˜4,700 bp), a 228 bp truncated miniature CMV promoter (mCMV), and a 16 bp soluble neuropilin-1 (sNRP-1) Poly A signal were used in these constructs, resulting in the 4,75 0 bp and 4,812 bp vector genome respectively (FIG. 7). These plasmids are used to produce scAAV-mCMV-hHGSNAT^{zc} and scAAV-mCMV-hHGSNAT viral vectors.

To determine whether the ptrsk-mCMV-hHGSNAT vector constructs generate scAAV vector, 3-plasmid-cotransfection was performed in HEK293 cells to produce scAAV9-CMV-hHGSNAT^{ZC} and scAAV9-CMV-hHGSNAT. As controls, pTR-CMV-hHGSNAT constructs were used to produce ssAAV-hHGSNAT vectors. The purified vector products were analyzed by alkaline denaturing gel electrophoresis. The results showed that the AAV-mCMV-hHGSNAT^{ZC} and AAV-mCMV-hHGSNAT viral vector products were scAAV vectors (FIG. 8). In contrast, the control AAV-CMV-hHGSNAT^{ZC} and AAV-CMV-hHGSNAT viral vector products were single-stranded AAV vectors, based on the mobility of the viral genome on the alkaline denaturing gel. These results indicate that using small mCMV promotor and SNRP-1 Poly A signal enabled the vg being packaged into scAAV-mCMV-hHGSNAT^{ZC} and scAAV-mCMV-hHGSNAT^{ZC} vectors.

### Example 2

### SMA Studies

The disclosed rAAV product was constructed by incorporation of EV-mRNA packaging signal to a scAAV-hSMN1 gene replacement vector to facilitate the bystander effect. The vector construct was tested using an in vitro model of MPS IIIC, to assess its therapeutic potential.

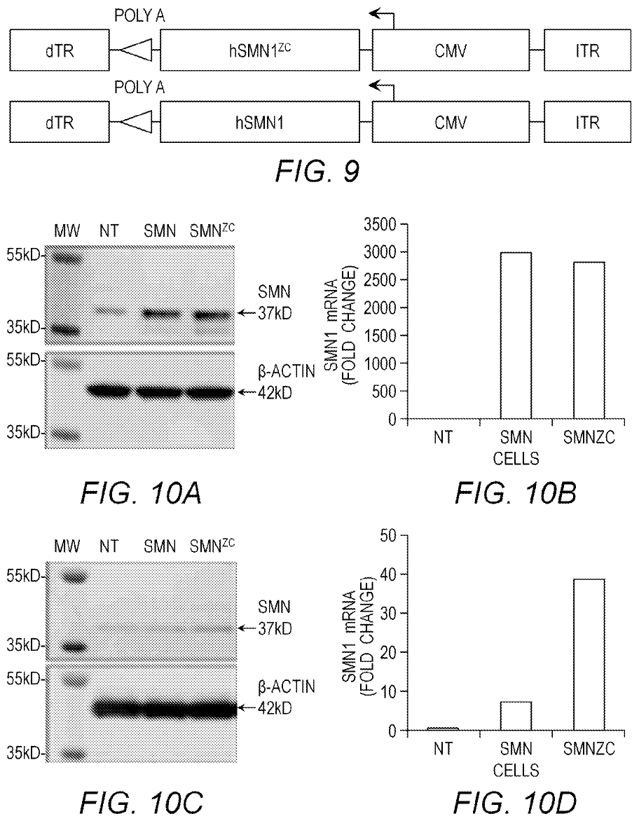

Construction of rAAV-hSMN1 vectors: A rAAV vector plasmid, ptrsk-CMV-hSMN1^{zc} (SEQ ID NO:6), was constructed to express hSMN protein, containing a 25 nt EV zip-code (ZC) signal sequence for packaging hSMN1-mRNA into EVs. A second AAV vector plasmid, ptrsk-CMV-hSMN1 (SEQ ID NO:7), without the EV signal, was constructed as a control. These plasmids are used to produce scAAV-CMV-hSMN1^{zc} and scAAV-CMV-hSMN1 viral vectors. The rAAV-hSMN1 viral vector genome (vg) contains only minimal elements required for transgene expression (FIG. 9).

rAAV-mediated expression of rSMN and functional EV-hSMN1-mRNA cargo in vitro in human SMN1 skin fibroblasts: To determine the function of these scAAV-hSMN1 vector constructs, human SMN1 skin fibroblasts (GM00232) were transfected with the vector plasmids (FIGS. 10A-10B), or incubated with EVs extracted from the vector plasmid transfected cells (FIGS. 10C-10D). At 48 h post transfection or 24 h post EV incubation, cell samples were assayed for SMN expression by western blot and qRT-PCR. Higher levels of SMN1 protein and SMN1 mRNA were detected in cells transfected with both vector plasmids (FIGS. 10A-10B). Increases in SMN1 protein and SMN1 mRNA were also detected in cells that were incubated with the EVs extracted from the media of hSMN120-transfected cells (FIGS. 10C-10D). These data demonstrate that both the hSMN12 and hSMN1 vectors mediated efficient rSMN expression, and importantly, EV-SMN1-mRNA cargo, leading to the translation of SMN1-mRNA to rSMN protein in recipient SMA cells. These data support the hypothesis on the potential of EV-facilitated bystander effect that may lead to the cross-correction of SMN defects in non-transduced cells. This bystander effect will allow the maximal therapeutic benefits from a relatively small number of transduced cells within a tissue, as long as they are well distributed, without having to transduce 100% of the cells. It may therefore significantly reduce the vector dose needed for optimal therapeutic efficacy for treating SMN in humans, and ease one of the critical challenges in translation, the scale-up manufacturing.

In conclusion, the novel AAV-SMN1 vector with the 25 nt EV ZC will mediate the bystander effect of rSMN1 via EV-rSMN1-mRNA cargo and significantly improve the efficacy of AAV gene replacement therapy for treating SMA. This rAAV mediated EV-mRNA cargo approach is applicable for gene replacement therapy treating many neurogenetic diseases involving a non-secreted protein.

### REFERENCES

1. Andrade, F., Aldamiz-Echevarria, L., Llarena, M. & Couce, M. L. Sanfilippo syndrome: Overall review. Pediatr Int 57, 331-8 (2015).
2. Lavery, C., Hendriksz, C. J. & Jones, S. A. Mortality in patients with Sanfilippo syndrome. Orphanet J Rare Dis 12, 168 (2017).
3. Foust, K. D. et al. Intravascular AAV9 preferentially targets neonatal neurons and adult astrocytes. Nat Biotechnol 27, 59-65 (2009).
4. Zincarelli, C., Soltys, S., Rengo, G. & Rabinowitz, J. E. Analysis of AAV serotypes 1-9 mediated gene expression and tropism in mice after systemic injection. Mol Ther 16, 1073-80 (2008).
5. Duque, S. et al. Intravenous administration of self-complementary AAV9 enables transgene delivery to adult motor neurons. Mol Ther 17, 1187-96 (2009).
6. Mendell, J. R. et al. Single-Dose Gene-Replacement Therapy for Spinal Muscular Atrophy. N Engl J Med 377, 1713-1722 (2017).
7. Fu, H. et al. Functional correction of neurological and somatic disorders at later stages of disease in MPS IIIA mice by systemic scAAV9-hSGSH gene delivery. Mol Ther Methods Clin Dev 3, 16036 (2016).
8. Fu, H., Dirosario, J., Killedar, S., Zaraspe, K. & McCarty, D. M. Correction of neurological disease of mucopolysaccharidosis IIIB in adult mice by rAAV9 trans-blood-brain barrier gene delivery. Mol Ther 19, 1025-33 (2011).
9. Fu, H. et al. Targeting Root Cause by Systemic scAAV9-hIDS Gene Delivery: Functional Correction and Reversal of Severe MPS II in Mice. Mol Ther Methods Clin Dev 10, 327-340 (2018).
10. Bobo, T. A., Samowitz, P. N., Robinson, M. I. & Fu, H. Targeting the root cause of mucopolysaccharidosis IIIA with a new scAAV9 gene replacement vector. Mol Ther—Methods & Clin Dev.
11. Lefebvre, S. et al. Identification and characterization of a spinal muscular atrophy-determining gene. Cell 80, 155-65 (1995).
12. Burghes, A. H. & Beattie, C. E. Spinal muscular atrophy: why do low levels of survival motor neuron protein make motor neurons sick? Nat Rev Neurosci 10, 597-609 (2009).
13. Coovert, D. D. et al. The survival motor neuron protein in spinal muscular atrophy. Hum Mol Genet 6, 1205-14 (1997).
14. Lefebvre, S. et al. Correlation between severity and SMN protein level in spinal muscular atrophy. Nat Genet 16, 265-9 (1997).
15. Pearn, J. Incidence, prevalence, and gene frequency studies of chronic childhood spinal muscular atrophy. J Med Genet 15, 409-13 (1978).
16. Sugarman, E. A. et al. Pan-ethnic carrier screening and prenatal diagnosis for spinal muscular atrophy: clinical laboratory analysis of >72,400 specimens. Eur J Hum Genet 20, 27-32 (2012).
17. Prior, T. W. et al. Newborn and carrier screening for spinal muscular atrophy. Am J Med Genet A 152A, 1608-16 (2010).
18. Schrank, B. et al. Inactivation of the survival motor neuron gene, a candidate gene for human spinal muscular atrophy, leads to massive cell death in early mouse embryos. Proc Natl Acad Sci USA 94, 9920-5 (1997).
19. Briese, M. et al. Deletion of smn-1, the Caenorhabditis elegans ortholog of the spinal muscular atrophy gene, results in locomotor dysfunction and reduced lifespan. Hum Mol Genet 18, 97-104 (2009).
20. Chan, Y. B. et al. Neuromuscular defects in a Drosophila survival motor neuron gene mutant. Hum Mol Genet 12, 1367-76 (2003).
21. Lorson, C. L., Hahnen, E., Androphy, E. J. & Wirth, B. A single nucleotide in the SMN gene regulates splicing and is responsible for spinal muscular atrophy. Proc Natl Acad Sci USA 96, 6307-11 (1999).
22. Monani, U. R. et al. A single nucleotide difference that alters splicing patterns distinguishes the SMA gene SMN1 from the copy gene SMN2. Hum Mol Genet 8, 1177-83 (1999).
23. Kashima, T. & Manley, J. L. A negative element in SMN2 exon 7 inhibits splicing in spinal muscular atrophy. Nat Genet 34, 460-3 (2003).
24. Gennarelli, M. et al. Survival motor neuron gene transcript analysis in muscles from spinal muscular atrophy patients. Biochem Biophys Res Commun 213, 342-8 (1995).

25 Arnold, W. D. & Burghes, A. H. Spinal muscular atrophy: development and implementation of potential treatments. Ann Neurol 74, 348-62 (2013).
26. Prior, T. W., Leach, M. E. & Finanger, E. Spinal Muscular Atrophy. in GeneReviews ((R)) (eds. Adam, M. P. et al.) (Seattle (WA), 1993).
27 Hausmanowa-Petrusewicz, I. & Karwanska, A. Electromyographic findings in different forms of infantile and juvenile proximal spinal muscular atrophy. Muscle Nerve 9, 37-46 (1986).
28. Buchthal, F. & Olsen, P. Z. Electromyography and muscle biopsy in infantile spinal muscular atrophy. Brain 93, 15-30 (1970).
29 Crawford, T. O. & Pardo, C. A. The neurobiology of childhood spinal muscular atrophy. Neurobiol Dis 3, 97-110 (1996).
30. Kolb, S. J. et al. Natural history of infantile-onset spinal muscular atrophy. Ann Neurol 82, 883-891 (2017).
31. Hache, M. et al. Intrathecal Injections in Children With Spinal Muscular Atrophy: Nusinersen Clinical Trial Experience. J Child Neurol 31, 899-906 (2016).
32. Hua, Y. et al. Antisense correction of SMN2 splicing in the CNS rescues necrosis in a type III SMA mouse model. Genes Dev 24, 1634-44 (2010).
33. Chiriboga, C. A. et al. Results from a phase 1 study of nusinersen (ISIS-SMN (Rx)) in children with spinal muscular atrophy. Neurology 86, 890-7 (2016).
34. Kolb, S. J. et al. Baseline results of the NeuroNEXT spinal muscular atrophy infant biomarker study. Ann Clin Transl Neurol 3, 132-45 (2016).
35. Naryshkin, N. A. et al. Motor neuron disease. SMN2 splicing modifiers improve motor function and longevity in mice with spinal muscular atrophy. Science 345, 688-93 (2014).
36. Poirier, A. et al. Risdiplam distributes and increases SMN protein in both the central nervous system and peripheral organs. Pharmacol Res Perspect 6, e00447 (2018).
37. Ratni, H. et al. Discovery of Risdiplam, a Selective Survival of Motor Neuron-2 (SMN2) Gene Splicing Modifier for the Treatment of Spinal Muscular Atrophy (SMA). J Med Chem 61, 6501-6517 (2018).
38. Hua, Y. et al. Peripheral SMN restoration is essential for long-term rescue of a severe spinal muscular atrophy mouse model. Nature 478, 123-6 (2011).
39. Urbanelli, L., Buratta, S., Sagini, K., Tancini, B. & Emiliani, C. Extracellular Vesicles as New Players in Cellular Senescence. Int J Mol Sci 17 (2016).
40. Meldolesi, J. Extracellular vesicles, news about their role in immune cells: physiology, pathology and diseases. Clin Exp Immunol 196, 318-327 (2019).
41. Valadi, H. et al. Exosome-mediated transfer of mRNAs and microRNAs is a novel mechanism of genetic exchange between cells. Nat Cell Biol 9, 654-9 (2007).
42. Colombo, M., Raposo, G. & Thery, C. Biogenesis, secretion, and intercellular interactions of exosomes and other extracellular vesicles. Annu Rev Cell Dev Biol 30, 255-89 (2014).
43. Belting, M. & Wittrup, A. Nanotubes, exosomes, and nucleic acid-binding peptides provide novel mechanisms of intercellular communication in eukaryotic cells: implications in health and disease. J Cell Biol 183, 1187-91 (2008).
44. Vlassov, A. V., Magdaleno, S., Setterquist, R. & Conrad, R. Exosomes: current knowledge of their composition, biological functions, and diagnostic and therapeutic potentials. Biochim Biophys Acta 1820, 940-8 (2012).
45. Mittelbrunn, M. & Sanchez-Madrid, F. Intercellular communication: diverse structures for exchange of genetic information. Nat Rev Mol Cell Biol 13, 328-35 (2012).
46. Hedlund, M., Nagaeva, O., Kargl, D., Baranov, V. & Mincheva-Nilsson, L. Thermal- and oxidative stress causes enhanced release of NKG2D ligand-bearing immunosuppressive exosomes in leukemia/lymphoma T and B cells. PLOS One 6, e16899 (2011).
47. Montecalvo, A. et al. Mechanism of transfer of functional microRNAs between mouse dendritic cells via exosomes. Blood 119, 756-66 (2012).
48. Li, J. et al. Exosomes mediate the cell-to-cell transmission of IFN-alpha-induced antiviral activity. Nat Immunol 14, 793-803 (2013).
49. Fatima, F. & Nawaz, M. Long Distance Metabolic Regulation through Adipose-Derived Circulating Exosomal miRNAs: A Trail for RNA-Based Therapies? Front Physiol 8, 545 (2017).
50. Li, X. et al. Challenges and opportunities in exosome research-Perspectives from biology, engineering, and cancer therapy. API. Bioeng 3, 011503 (2019).
51. Mizrak, A. et al. Genetically engineered microvesicles carrying suicide mRNA/protein inhibit schwannoma tumor growth. Mol Ther 21, 101-8 (2013).
52. Barile, L. & Vassalli, G. Exosomes: Therapy delivery tools and biomarkers of diseases. Pharmacol Ther 174, 63-78 (2017).
53. Robbins, P. D. & Morelli, A. E. Regulation of immune responses by extracellular vesicles. Nat Rev Immunol 14, 195-208 (2014).
54. Saenz-Cuesta, M. et al. Circulating microparticles reflect treatment effects and clinical status in multiple sclerosis. Biomark Med 8, 653-61 (2014).
55. Logozzi, M. et al. High levels of exosomes expressing CD63 and caveolin-1 in plasma of melanoma patients. PLOS One 4, e5219 (2009).
56. de Jong, O. G. et al. Drug Delivery with Extracellular Vesicles: From Imagination to Innovation. Acc Chem Res (2019).
57. Turchinovich, A., Drapkina, O. & Tonevitsky, A. Transcriptome of Extracellular Vesicles: State-of-the-Art. Front Immunol 10, 202 (2019).
58. Lasser, C. et al. Two distinct extracellular RNA signatures released by a single cell type identified by microarray and next-generation sequencing. RNA Biol 14, 58-72 (2017).
59. Bolukbasi, M. F. et al. miR-1289 and “Zipcode”-like Sequence Enrich mRNAs in Microvesicles. Mol Ther Nucleic Acids 1, e10 (2012).
60. Statello, L. et al. Identification of RNA-binding proteins in exosomes capable of interacting with different types of RNA: RBP-facilitated transport of RNAs into exosomes. PLOS One 13, e0195969 (2018).
61. Villarroya-Beltri, C. et al. Sumoylated hnRNPA2B1 controls the sorting of miRNAs into exosomes through binding to specific motifs. Nat Commun 4, 2980 (2013).
62. Wu, B. et al. Molecular basis for the specific and multivariant recognitions of RNA substrates by human hnRNP A2/B1. Nat Commun 9, 420 (2018).
63. Kossinova, O. A. et al. Cytosolic YB-1 and NSUN2 are the only proteins recognizing specific motifs present in mRNAs enriched in exosomes. Biochim Biophys Acta Proteins Proteom 1865, 664-673 (2017).
64. Shurtleff, M. J. et al. Broad role for YBX1 in defining the small noncoding RNA composition of exosomes. Proc Natl Acad Sci USA 114, E8987-E8995 (2017).
65. Yanshina, D. D. et al. Structural features of the interaction of the 3′-untranslated region of mRNA containing exosomal RNA-specific motifs with YB-1, a potential mediator of mRNA sorting. Biochimie 144, 134-143 (2018).

The foregoing examples are illustrative of the present invention and are not to be construed as limiting thereof. Although the invention has been described in detail with reference to preferred embodiments, variations and modifications exist within the scope and spirit of the invention as described and defined in the following claims.

Sequences

Extracellular vesicle-targeting zip code sequence (SEQ ID NO: 1)
ACCCTGCCGCCTGGACTCCGCCTGT
pTR-CMV-hHGSNAT^{zc} (SEQ ID NO: 2)
GGGGGGGGGGGGGGGGGGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAGAGAGGGA
GTGGCCAACTCCATCACTAGGGGTTCCTAGATCTGAATTCGGTACCCGTTACATAACTTACGGTAAATG
GCCCGCCTGGCTGACCGCCCAACGACCCCCGCCCATTGACGTCAATAATGACGTATGTTCCCATAGTA
ACGCCAATAGGGACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCATCGCTA
TTACCATGGTGATGCGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAACGGGACTTTCCAAA
ATGTCGTAACAACTCCGCCCCATTGACGCAAATGGGCGGTAGGCGTGTACGGTGGGAGGTCTATATAA
GCAGAGCTCGTTTAGTGAACCGTCAGATCGCCTGGAGACGCCATCCACGCTGTTTTGACCTCCATAGA
AGACACCGGGACCGATCCAGCCTCCGGACTCTAGAATGAGCGGGGGGGGCAGGGCGCTGGCCGCGCT
GCTGCTGGCCGCGTCCGTGCTGAGCGCCGCGCTGCTGGCCCCCGGCGGCTCTTCGGGGCGCGATGCCC
AGGCCGCGCCGCCACGAGACTTAGACAAAAAAAGACATGCAGAGCTGAAGATGGATCAGGCTTTGCT
ACTCATCCATAATGAACTTCTCTGGACCAACTTGACCGTCTACTGGAAATCTGAATGCTGTTATCACTG
CTTGTTTCAGGTTCTGGTAAACGTTCCTCAGAGTCCAAAAGCAGGGAAGCCTAGTGCTGCAGCTGCCT
CTGTCAGCACCCAGCACGGATCTATCCTGCAGCTGAACGACACCTTGGAAGAGAAAGAAGTTTGTAGG
TTGGAATACAGATTTGGAGAATTTGGAAACTATTCTCTCTTGGTAAAGAACATCCATAATGGAGTTAG
TGAAATTGCCTGTGACCTGGCTGTGAACGAGGATCCAGTTGATAGTAACCTTCCTGTGAGCATTGCATT
CCTTATTGGTCTTGCTGTCATCATTGTGATATCCTTTCTGAGGCTCTTGTTGAGTTTGGATGACTTTAAC
AATTGGATTTCTAAAGCCATAAGTTCTCGAGAAACTGATCGCCTCATCAATTCTGAGCTGGGATCTCCC
AGCAGGACAGACCCTCTCGATGGTGATGTTCAGCCAGCAACGTGGCGTCTATCTGCCCTGCCGCCCCG
CCTCCGCAGCGTGGACACCTTCAGGGGGATTGCTCTTATACTCATGGTCTTTGTCAATTATGGAGGAGG
AAAATATTGGTACTTCAAACATGCAAGTTGGAATGGGCTGACAGTGGCTGACCTCGTGTTCCCGTGGT
TTGTATTTATTATGGGATCTTCCATTTTTCTATCGATGACTTCTATACTGCAACGGGGGTGTTCAAAATT
CAGATTGCTGGGGAAGATTGCATGGAGGAGTTTCCTGTTAATCTGCATAGGAATTATCATTGTGAATC
CCAATTATTGCCTTGGTCCATTGTCTTGGGACAAGGTGCGCATTCCTGGTGTGCTGCAGCGATTGGGAG
TGACATACTTTGTGGTTGCTGTGTTGGAGCTCCTCTTTGCTAAACCTGTGCCTGAACATTGTGCCTCGG
AGAGGAGCTGCCTTTCTCTTCGAGACATCACGTCCAGCTGGCCCCAGTGGCTGCTCATCCTGGTGCTGG
AAGGCCTGTGGCTGGGCTTGACATTCCTCCTGCCAGTCCCTGGGTGCCCTACTGGTTATCTTGGTCCTG
GGGGCATTGGAGATTTTGGCAAGTATCCAAATTGCACTGGAGGAGCTGCAGGCTACATCGACCGCCTG
CTGCTGGGAGACGATCACCTTTACCAGCACCCATCTTCTGCTGTACTTTACCACACCGAGGTGGCCTAT
GACCCCGAGGGCATCCTGGGCACCATCAACTCCATCGTGATGGCCTTTTTAGGAGTTCAGGCAGGAAA
AATACTATTGTATTACAAGGCTCGGACCAAAGACATCCTGATTCGATTCACTGCTTGGTGTTGTATTCT
TGGGCTCATTTCTGTTGCTCTGACGAAGGTTTCTGAAAATGAAGGCTTTATTCCAGTAAACAAAAATCT
CTGGTCCCTTTCGTATGTCACTACGCTCAGTTCTTTTGCCTTCTTCATCCTGCTGGTCCTGTACCCAGTT
GTGGATGTGAAGGGGCTGTGGACAGGAACCCCATTCTTTTATCCAGGAATGAATTCCATTCTGGTATA
TGTCGGCCACGAGGTGTTTGAGAACTACTTCCCCTTTCAGTGGAAGCTGAAGGACAACCAGTCCCACA
AGGAGCACCTGACTCAGAACATCGTCGCCACTGCCCTCTGGGTGCTCATTGCCTACATCCTCTATAGAA
AGAAGATTTTTTGGAAAATCTGAGTCGACACCCTGCCGCCTGGACTCCGCCTGTTAGAGCTCGCTGATC
AGCCTCGACTGTGCCTTCTAGTTGCCAGCCATCTGTTGTTTGCCCCTCCCCCGTGCCTTCCTTGACCCTG
GAAGGTGCCACTCCCACTGTCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTGGGAAGACAATAGCAGGC
ATGCTGGGGAGAGATCTAGGAACCCCTAGTGATGGAGTTGGCCACTCCCTCTCTGCGCGCTCGCTCGC
TCACTGAGGCCGCCCGGGCAAAGCCCGGGCGTCGGGCGACCTTTGGTCGCCCGGCCTCAGTGAGCGAG
CGAGCGCGCAGAGAGGGAGTGGCCAACCCCCCCCCCCCCCCCCCTGCAGCCCAGCTGCATTAATGAAT
CGGCCAACGCGCGGGGAGAGGCGGTTTGCGTATTGGGCGCTCTTCCGCTTCCTCGCTCACTGACTCGCT
GCGCTCGGTCGTTCGGCTGCGGCGAGCGGTATCAGCTCACTCAAAGGCGGTAATACGGTTATCCACAG
AATCAGGGGATAACGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAAAAATCGACGCTCA
AGTCAGAGGTGGCGAAACCCGACAGGACTATAAAGATACCAGGCGTTTCCCCCTGGAAGCTCCCTCGT
GCGCTCTCCTGTTCCGACCCTGCCGCTTACCGGATACCTGTCCGCCTTTCTCCCTTCGGGAAGCGTGGC
GCTTTCTCAATGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGTATGTAGGC
GGTGCTACAGAGTTCTTGAAGTGGTGGCCTAACTACGGCTACACTAGAAGGACAGTATTTGGTATCTG
CGCTCTGCTGAAGCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGATCTCAAGAAGAT
CCTTTGATCTTTTCTACGGGGTCTGACGCTCAGTGGAACGAAAACTCACGTTAAGGGATTTTGGTCATG
AGATTATCAAAAAGGATCTTCACCTAGATCCTTTTAAATTAAAAATGAAGTTTTAAATCAATCTAAAGT
ATATATGAGTAAACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGGAGGGCTTACCA
TCTGGCCCCAGTGCTGCAATGATACCGCGAGACCCACGCTCACCGGCTCCAGATTTATCAGCAATAAA
CCAGCCAGCCGGAAGGGCCGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCCGGTTCCCAACGATCAAGGC
GAGTTACATGATCCCCCATGTTGTGCAAAAAAGCGGTTAGCTCCTTCGGTCCTCCGATCGTTGTCAGAA
GTAAGTTGGCCGCAGTGTTATCACTCATGGTTATGGCAGCACTGCATAATTCTCTTACTGTCATGCCAT
CCGTAAGATGCTTTTCTGTGACTGGTGAGTACTCAACCAAGTCATTCTGAGAATAGTGTATGCGGCGA
CCGAGTTGCTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATCCAGTTCGA
TGTAACCCACTCGTGCACCCAACTGATCTTCAGCATCTTTTACTTTCACCAGCGTTTCTGGGTGAGCAA
AAACAGGAAGGCAAAATGCCGCAAAAAAGGGAATAAGGGCGACACGGAAATGTTGAATACTCATACT
CTTCCTTTTTCAATATTATTGAAGCATTTATCAGGGTTATTGTCTCATGAGCGGATACATATTTGAATGT
ATTTAGAAAAATAAACAAATAGGGGTTCCGCGCACATTTCCCCGAAAAGTGCCACCTGACGTCTAAGA
AACCATTATTATCATGACATTAACCTATAAAAATAGGCGTATCACGAGGCCCTTTCGTCTCGCGCGTTT
CGGTGATGACGGTGAAAACCTCTGACACATGCAGCTCCCGGAGACGGTCACAGCTTGTCTGTAAGCGG
ATGCCGGGAGCAGACAAGCCCGTCAGGGCGCGTCAGCGGGTGTTGGCGGGTGTCGGGGCTGGCTTAA
CTATGCGGCATCAGAGCAGATTGTACTGAGAGTGCACCATATGCGGTGTGAAATACCGCACAGATGCG
TAAGGAGAAAATACCGCATCAGGAAATTGTAAACGTTAATATTTTGTTAAAATTCGCGTTAAATTTTTG
TTAAATCAGCTCATTTTTTAACCAATAGGCCGAAATCGGCAAAATCCCTTATAAATCAAAAGAATAGA
CCGAGATAGGGTTGAGTGTTGTTCCAGTTTGGAACAAGAGTCCACTATTAAAGAACGTGGACTCCAAC
GTCAAAGGGCGAAAAACCGTCTATCAGGGCGATGGCCCACTACGTGAACCATCACCCTAATCAAGTTT
TTTGGGGTCGAGGTGCCGTAAAGCACTAAATCGGAACCCTAAAGGGAGCCCCCGATTTAGAGCTTGAC
GGGGAAAGCCGGCGAACGTGGCGAGAAAGGAAGGGAAGAAAGCGAAAGGAGCGGGCGCTAGGGCG
CTGGCAAGTGTAGCGGTCACGCTGCGCGTAACCACCACACCCGCCGCGCTTAATGCGCCGCTACAGGG
CGCGTCGCGCCATTCGCCATTCAGGCTACGCAACTGTTGGGAAGGGCGATCGGTGCGGGCCTCTTCGC
TATTACGCCAGCTGGCTGCA
pTR-CMV-hHGSNAT (SEQ ID NO: 3)
GGGGGGGGGGGGGGGGGGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAGAGAGGGA
GTGGCCAACTCCATCACTAGGGGTTCCTAGATCTGAATTCGGTACCCGTTACATAACTTACGGTAAATG
GCCCGCCTGGCTGACCGCCCAACGACCCCCGCCCATTGACGTCAATAATGACGTATGTTCCCATAGTA
ACGCCAATAGGGACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCATCGCTA
TTACCATGGTGATGCGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAACGGGACTTTCCAAA
ATGTCGTAACAACTCCGCCCCATTGACGCAAATGGGCGGTAGGCGTGTACGGTGGGAGGTCTATATAA
GCAGAGCTCGTTTAGTGAACCGTCAGATCGCCTGGAGACGCCATCCACGCTGTTTTGACCTCCATAGA
AGACACCGGGACCGATCCAGCCTCCGGACTCTAGAATGAGCGGGGGGGGCAGGGCGCTGGCCGCGCT
GCTGCTGGCCGCGTCCGTGCTGAGCGCCGCGCTGCTGGCCCCCGGCGGCTCTTCGGGGCGCGATGCCC
AGGCCGCGCCGCCACGAGACTTAGACAAAAAAAGACATGCAGAGCTGAAGATGGATCAGGCTTTGCT
ACTCATCCATAATGAACTTCTCTGGACCAACTTGACCGTCTACTGGAAATCTGAATGCTGTTATCACTG
CTTGTTTCAGGTTCTGGTAAACGTTCCTCAGAGTCCAAAAGCAGGGAAGCCTAGTGCTGCAGCTGCCT
CTGTCAGCACCCAGCACGGATCTATCCTGCAGCTGAACGACACCTTGGAAGAGAAAGAAGTTTGTAGG
TTGGAATACAGATTTGGAGAATTTGGAAACTATTCTCTCTTGGTAAAGAACATCCATAATGGAGTTAG
TGAAATTGCCTGTGACCTGGCTGTGAACGAGGATCCAGTTGATAGTAACCTTCCTGTGAGCATTGCATT
CCTTATTGGTCTTGCTGTCATCATTGTGATATCCTTTCTGAGGCTCTTGTTGAGTTTGGATGACTTTAAC
AATTGGATTTCTAAAGCCATAAGTTCTCGAGAAACTGATCGCCTCATCAATTCTGAGCTGGGATCTCCC
AGCAGGACAGACCCTCTCGATGGTGATGTTCAGCCAGCAACGTGGCGTCTATCTGCCCTGCCGCCCCG
CCTCCGCAGCGTGGACACCTTCAGGGGGATTGCTCTTATACTCATGGTCTTTGTCAATTATGGAGGAGG
AAAATATTGGTACTTCAAACATGCAAGTTGGAATGGGCTGACAGTGGCTGACCTCGTGTTCCCGTGGT
TTGTATTTATTATGGGATCTTCCATTTTTCTATCGATGACTTCTATACTGCAACGGGGGTGTTCAAAATT
CAGATTGCTGGGGAAGATTGCATGGAGGAGTTTCCTGTTAATCTGCATAGGAATTATCATTGTGAATC
CCAATTATTGCCTTGGTCCATTGTCTTGGGACAAGGTGCGCATTCCTGGTGTGCTGCAGCGATTGGGAG
TGACATACTTTGTGGTTGCTGTGTTGGAGCTCCTCTTTGCTAAACCTGTGCCTGAACATTGTGCCTCGG
AGAGGAGCTGCCTTTCTCTTCGAGACATCACGTCCAGCTGGCCCCAGTGGCTGCTCATCCTGGTGCTGG
AAGGCCTGTGGCTGGGCTTGACATTCCTCCTGCCAGTCCCTGGGTGCCCTACTGGTTATCTTGGTCCTG
GGGGCATTGGAGATTTTGGCAAGTATCCAAATTGCACTGGAGGAGCTGCAGGCTACATCGACCGCCTG
CTGCTGGGAGACGATCACCTTTACCAGCACCCATCTTCTGCTGTACTTTACCACACCGAGGTGGCCTAT
GACCCCGAGGGCATCCTGGGCACCATCAACTCCATCGTGATGGCCTTTTTAGGAGTTCAGGCAGGAAA
AATACTATTGTATTACAAGGCTCGGACCAAAGACATCCTGATTCGATTCACTGCTTGGTGTTGTATTCT
TGGGCTCATTTCTGTTGCTCTGACGAAGGTTTCTGAAAATGAAGGCTTTATTCCAGTAAACAAAAATCT
CTGGTCCCTTTCGTATGTCACTACGCTCAGTTCTTTTGCCTTCTTCATCCTGCTGGTCCTGTACCCAGTT
GTGGATGTGAAGGGGCTGTGGACAGGAACCCCATTCTTTTATCCAGGAATGAATTCCATTCTGGTATA
TGTCGGCCACGAGGTGTTTGAGAACTACTTCCCCTTTCAGTGGAAGCTGAAGGACAACCAGTCCCACA
AGGAGCACCTGACTCAGAACATCGTCGCCACTGCCCTCTGGGTGCTCATTGCCTACATCCTCTATAGAA
AGAAGATTTTTTGGAAAATCTGAGTCGACTAGAGCTCGCTGATCAGCCTCGACTGTGCCTTCTAGTTGC
CAGCCATCTGTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGAGAGATCTAGGAAC
CCCTAGTGATGGAGTTGGCCACTCCCTCTCTGCGCGCTCGCTCGCTCACTGAGGCCGCCCGGGCAAAG
CCCGGGCGTCGGGCGACCTTTGGTCGCCCGGCCTCAGTGAGCGAGCGAGCGCGCAGAGAGGGAGTGG
CCAACCCCCCCCCCCCCCCCCCTGCAGCCCAGCTGCATTAATGAATCGGCCAACGCGCGGGGAGAGGC
GGTTTGCGTATTGGGCGCTCTTCCGCTTCCTCGCTCACTGACTCGCTGCGCTCGGTCGTTCGGCTGCGG
CGAGCGGTATCAGCTCACTCAAAGGCGGTAATACGGTTATCCACAGAATCAGGGGATAACGCAGGAA
AGAACATGTGAGCAAAAGGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTCAGAGGTGGCGAAACCCG
ACAGGACTATAAAGATACCAGGCGTTTCCCCCTGGAAGCTCCCTCGTGCGCTCTCCTGTTCCGACCCTG
CCGCTTACCGGATACCTGTCCGCCTTTCTCCCTTCGGGAAGCGTGGCGCTTTCTCAATGCTCACGCTGT
AGGTATCTCAGTTCGGTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACACGACTTATCGCCACTG
GCAGCAGCCACTGGTAACAGGATTAGCAGAGCGAGGTATGTAGGCGGTGCTACAGAGTTCTTGAAGT
GGTGGCCTAACTACGGCTACACTAGAAGGACAGTATTTGGTATCTGCGCTCTGCTGAAGCCAGTTACC
TTCGGAAAAAGAGTTGGTAGCTCTTGATCCGGCAAACAAACCACCGCTGGTAGCGGTGGTTTTTTTGTT
TGCAAGCAGCAGATTACGCGCAGAAAAAAAGGATCTCAAGAAGATCCTTTGATCTTTTCTACGGGGTC
TGACGCTCAGTGGAACGAAAACTCACGTTAAGGGATTTTGGTCATGAGATTATCAAAAAGGATCTTCA
CCTAGATCCTTTTAAATTAAAAATGAAGTTTTAAATCAATCTAAAGTATATATGAGTAAACTTGGTCTG
ACAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCG
AGCGCAGAAGTGGTCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCATCGTGGTGTCACGC
TCGTCGTTTGGTATGGCTTCATTCAGCTCCGGTTCCCAACGATCAAGGCGAGTTACATGATCCCCCATG
TTGTGCAAAAAAGCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGATGCTTTTCTGTG
ACTGGTGAGTACTCAACCAAGTCATTCTGAGAATAGTGTATGCGGCGACCGAGTTGCTCTTGCCCGGC
GTCAATACGGGATAATACCGCGCCACATAGCAGAACTTTAAAAGTGCTCATCATTGGAAAACGTTCTT
CGGGGCGAAAACTCTCAAGGATCTTACCGCTGTTGAGATCCAGTTCGATGTAACCCACTCGTGCACCC
AACTGATCTTCAGCATCTTTTACTTTCACCAGCGTTTCTGGGTGAGCAAAAACAGGAAGGCAAAATGC
CGCAAAAAAGGGAATAAGGGCGACACGGAAATGTTGAATACTCATACTCTTCCTTTTTCAATATTATT
GAAGCATTTATCAGGGTTATTGTCTCATGAGCGGATACATATTTGAATGTATTTAGAAAAATAAACAA
ATAGGGGTTCCGCGCACATTTCCCCGAAAAGTGCCACCTGACGTCTAAGAAACCATTATTATCATGAC
ATTAACCTATAAAAATAGGCGTATCACGAGGCCCTTTCGTCTCGCGCGTTTCGGTGATGACGGTGAAA
ACCTCTGACACATGCAGCTCCCGGAGACGGTCACAGCTTGTCTGTAAGCGGATGCCGGGAGCAGACAA
GCCCGTCAGGGCGCGTCAGCGGGTGTTGGCGGGTGTCGGGGCTGGCTTAACTATGCGGCATCAGAGCA
GATTGTACTGAGAGTGCACCATATGCGGTGTGAAATACCGCACAGATGCGTAAGGAGAAAATACCGC
ATCAGGAAATTGTAAACGTTAATATTTTGTTAAAATTCGCGTTAAATTTTTGTTAAATCAGCTCATTTTT
TAACCAATAGGCCGAAATCGGCAAAATCCCTTATAAATCAAAAGAATAGACCGAGATAGGGTTGAGT
GTTGTTCCAGTTTGGAACAAGAGTCCACTATTAAAGAACGTGGACTCCAACGTCAAAGGGCGAAAAAC
CGTCTATCAGGGCGATGGCCCACTACGTGAACCATCACCCTAATCAAGTTTTTTGGGGTCGAGGTGCC
GTAAAGCACTAAATCGGAACCCTAAAGGGAGCCCCCGATTTAGAGCTTGACGGGGAAAGCCGGCGAA
CGTGGCGAGAAAGGAAGGGAAGAAAGCGAAAGGAGCGGGCGCTAGGGCGCTGGCAAGTGTAGCGGT
CACGCTGCGCGTAACCACCACACCCGCCGCGCTTAATGCGCCGCTACAGGGCGCGTCGCGCCATTCGC
CATTCAGGCTACGCAACTGTTGGGAAGGGCGATCGGTGCGGGCCTCTTCGCTATTACGCCAGCTGGCT
GCA
ptrsk-mCMV-hHGSNAT^{ZC} (SEQ ID NO: 4)
CATTAATGAATCGGCCAACGCGCGGGGAGAGGCGGTTTGCGTATTGGGCGCTCTTCCGCTTCCTCGCT
CACTGACTCGCTGCGCTCGGTCGTTCGGCTGCGGCGAGCGGTATCAGCTCACTCAAAGGCGGTAATAC
GGTTATCCACAGAATCAGGGGATAACGCAGGAAAGAACATGTGAGCAAAAGGCCAGCAAAAGGCCA
GGAACCGTAAAAAGGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACCTGTCCGCCTTTCTCCCTTC
GGGAAGCGTGGCGCTTTCTCATAGCTCACGCTGTAGGTATCTCAGTTCGGTGTAGGTCGTTCGCTCCAA
GCTGGGCTGTGTGCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGATTAGCAGAGCG
AGGTATGTAGGCGGTGCTACAGAGTTCTTGAAGTGGTGGCCTAACTACGGCTACACTAGAAGAACAGT
ATTTGGTATCTGCGCTCTGCTGAAGCCAGTTACCTTCGGAAAAAGAGTTGGTAGCTCTTGATCCGGCAA
ACAAACCACCGCTGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCAGTGGAACGAAAACTCACGTTAAGGG
ATTTTGGTCATGAGATTATCAAAAAGGATCTTCACCTAGATCCTTTTAAATTAAAAATGAAGTTTTAAA
TCAATCTAAAGTATATATGAGTAAACTTGGTCTGACAGTTACCAATGCTTAATCAGTGAGGCACCTATC
TCAGCGATCTGTCTATTTCGTTCATCCATAGTTGCCTGACTCCCCGTCGTGTAGATAACTACGATACGG
GAGGGCTTACCATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTTATCCGCCTCCA
TCCAGTCTATTAATTGTTGCCGGGAAGCTAGAGTAAGTAGTTCGCCAGTTAATAGTTTGCGCAACGTTG
TTGCCATTGCTACAGGCATCGTGGTGTCACGCTCGTCGTTTGGTATGGCTTCATTCAGCTCCGGTTCCC
AACGATCAAGGCGAGTTACATGATCCCCCATGTTGTGCAAAAAAGCGGTTAGCTCCTTCGGTCCTCCG
ATCGTTGTCAGAAGTAAGTTGGCCGCAGTGTTATCACTCATGGTTATGGCAGCACTGCATAATTCTCTT
ACTGTCATGCCATCCGTAAGATGCTTTTCTGTGACTGGTGAGTACTCAACCAAGTCATTCTGAGAATAG
TGTATGCGGCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAAGTGCCAC
CTGACGTCTAAGAAACCATTATTATCATGACATTAACCTATAAAAATAGGCGTATCACGAGGCCCTTT
CGTCTCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGGTGTC
GGGGCTGGCTTAACTATGCGGCATCAGAGCAGATTGTACTGAGAGTGCACCATATGCGGTGTGAAATA
CCGCACAGATGCGTAAGGAGAAAATACCGCATCAGGCGATTCCAACATCCAATAAATCATACAGGCA
AGGCAAAGAATTAGCAAAATTAAGCAATAAAGCCTCAGAGCATAAAGCTAAATCGGTTGTACCAAAA
ACATTATGACCCTGTAATACTTTTGCGGGAGAAGCCTTTATTTCAACGCAAGGATAAAAATTTTTAGAA
CCCTCATATATTTTAAATGCAATGCCTGAGTAATGTGTAGGTAAAGATTCAAACGGGTGAGAAAGGCC
GGAGACAGTCAAATCACCATCAATATGATATTCAACCGTTCTAGCTGATAAATTCATGCCGGAGAGGG
TAGCTATTTTTGAGAGGTCTCTACAAAGGCTATCAGGTCATTGCCTGAGAGTCTGGAGCAAACAAGAG
AATCGATGAACGGTAATCGTAAAACTAGCATGTCAATCATATGTACCCCGGTTGATAATCAGAAAAGC
CCCAAAAACAGGAAGATTGTATAAGCAAATATTTAAATTGTAAGCGTTAATATTTTGTTAAAATTCGC
GTTAAATTTTTGTTAAATCAGCTCATTTTTTAACCAATAGGCCGAAATCGGCAAAATCCCTTATAAATC
AAAAGAATAGACCGAGATAGGGTTGAGTGTTGTTCCAGTTTGGAACAAGAGTCCACTATTAAAGAAC
GTGGACTCCAACGTCAAAGGGCGAAAAACCGTCTATCAGGGCGATGGCCCACTACGTGAACCATCAC
CCTAATCAAGTTTTTTGGGGTCGAGGTGCCGTAAAGCACTAAATCGGAACCCTAAAGGGAGCCCCCGA
TTTAGAGCTTGACGGGGAAAGCCGGCGAACGTGGCGAGAAAGGAAGGGAAGAAAGCGAAAGGAGCG
GGCGCTAGGGCGCTGGCAAGTGTAGCGGTCACGCTGCGCGTAACCACCACACCCGCCGCGCTTAATGC
GCCGCTACAGGGCGCGTACTATGGTTGCTTTGACGAGCACGTATAACGTGCTTTCCTCGTTAGAATCAG
AGCGGGAGCTAAACAGGAGGCCGATTAAAGGGATTTTAGACAGGAACGGTACGCCAGAATCCTGAGA
AGTGTTTTTATAATCAGTGAGGCCACCGAGTAAAAGAGTCTGTCCATCACGCAAATTAACCGTTGTCG
CAATACTTCTTTGATTAGTAATAACATCACTTGCCTGAGTAGAAGAACTCAAACTATCGGCCTTGCTGG
TAATATCCAGAACAATATTACCGCCAGCCATTGCAACGGAATCGCCATTCGCCATTCAGGCTGCGCAA
CTGTTGGGAAGGGCGATCGGTGCGGGCCTCTTCGCTATTACGCCAGCTGCGCGCTCGCTCGCTCACTG
AGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTAGGAAGCTTTCGTTACATAACTTACGG
TAAATGGCCCGCCTGGCTGACCGCCCAACGACCCCCGGACTCACGGGGATTTCCAAGTCTCCACCCCA
TTGACGTCAATGGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAGAGCTCGTTTAGT
GAACCGCTCGACCGGTCGCCATGAGCGGGGGGGGCAGGGCGCTGGCCGCGCTGCTGCTGGCCGCGTC
CGTGCTGAGCGCCGCGCTGCTGGCCCCCGGCGGCTCTTCGGGGCGCGATGCCCAGGCCGCGCCGCCAC
GAGACTTAGACAAAAAAAGACATGCAGAGCTGAAGATGGATCAGGCTTTGCTACTCATCCATAATGA
ACTTCTCTGGACCAACTTGACCGTCTACTGGAAATCTGAATGCTGTTATCACTGCTTGTTTCAGGTTCT
GGTAAACGTTCCTCAGAGTCCAAAAGCAGGGAAGCCTAGTGCTGCAGCTGCCTCTGTCAGCACCCAGC
ACGGATCTATCCTGCAGCTGAACGACACCTTGGAAGAGAAAGAAGTTTGTAGGTTGGAATACAGATTT
GGAGAATTTGGAAACTATTCTCTCTTGGTAAAGAACATCCATAATGGAGTTAGTGAAATTGCCTGTGA
CCTGGCTGTGAACGAGGATCCAGTTGATAGTAACCTTCCTGTGAGCATTGCATTCCTTATTGGTCTTGC
TGTCATCATTGTGATATCCTTTCTGAGGCTCTTGTTGAGTTTGGATGACTTTAACAATTGGATTTCTAAA
GCCATAAGTTCTCGAGAAACTGATCGCCTCATCAATTCTGAGCTGGGATCTCCCAGCAGGACAGACCC
TCTCGATGGTGATGTTCAGCCAGCAACGTGGCGTCTATCTGCCCTGCCGCCCCGCCTCCGCAGCGTGGA
CACCTTCAGGGGGATTGCTCTTATACTCATGGTCTTTGTCAATTATGGAGGAGGAAAATATTGGTACTT
CAAACATGCAAGTTGGAATGGGCTGACAGTGGCTGACCTCGTGTTCCCGTGGTTTGTATTTATTATGGG
ATCTTCCATTTTTCTATCGATGACTTCTATACTGCAACGGGGGTGTTCAAAATTCAGATTGCTGGGGAA
GATTGCATGGAGGAGTTTCCTGTTAATCTGCATAGGAATTATCATTGTGAATCCCAATTATTGCCTTGG
TCCATTGTCTTGGGACAAGGTGCGCATTCCTGGTGTGCTGCAGCGATTGGGAGTGACATACTTTGTGGT
TGCTGTGTTGGAGCTCCTCTTTGCTAAACCTGTGCCTGAACATTGTGCCTCGGAGAGGAGCTGCCTTTC
TCTTCGAGACATCACGTCCAGCTGGCCCCAGTGGCTGCTCATCCTGGTGCTGGAAGGCCTGTGGCTGG
GCTTGACATTCCTCCTGCCAGTCCCTGGGTGCCCTACTGGTTATCTTGGTCCTGGGGGCATTGGAGATT
TTGGCAAGTATCCAAATTGCACTGGAGGAGCTGCAGGCTACATCGACCGCCTGCTGCTGGGAGACGAT
CACCTTTACCAGCACCCATCTTCTGCTGTACTTTACCACACCGAGGTGGCCTATGACCCCGAGGGCATC
CTGGGCACCATCAACTCCATCGTGATGGCCTTTTTAGGAGTTCAGGCAGGAAAAATACTATTGTATTAC
AAGGCTCGGACCAAAGACATCCTGATTCGATTCACTGCTTGGTGTTGTATTCTTGGGCTCATTTCTGTT
GCTCTGACGAAGGTTTCTGAAAATGAAGGCTTTATTCCAGTAAACAAAAATCTCTGGTCCCTTTCGTAT
GTCACTACGCTCAGTTCTTTTGCCTTCTTCATCCTGCTGGTCCTGTACCCAGTTGTGGATGTGAAGGGG
CTGTGGACAGGAACCCCATTCTTTTATCCAGGAATGAATTCCATTCTGGTATATGTCGGCCACGAGGTG
TTTGAGAACTACTTCCCCTTTCAGTGGAAGCTGAAGGACAACCAGTCCCACAAGGAGCACCTGACTCA
GAACATCGTCGCCACTGCCCTCTGGGTGCTCATTGCCTACATCCTCTATAGAAAGAAGATTTTTTGGAA
AATCTGACGTCGACACCCTGCCGCCTGGACTCCGCCTGTCATCAGATCTAAATAAAATACGAAATG
TTAAACCCCACTCCCTCTCTGCGCGCTCGCTCGCTCACTGAGGCCGGGCGACCAAAGGTCGCCCGACG
CCCGGGCTTTGCCCGGGCGGCCTCAGTGAGCGAGCGAGCGCGCAGCTGCTG
ptrsk-mCMV-hHGSNAT (SEQ ID NO: 5)
CATTAATGAATCGGCCAACGCGCGGGGAGAGGCGGTTTGCGTATTGGGCGCTCTTCCGCTTCCTCGCT
CACTGACTCGCTGCGCTCGGTCGTTCGGCTGCGGCGAGCGGTATCAGCTCACTCAAAGGCGGTAATAC
GGTTATCCACAGAATCAGGGGATAACGCAGGAAAGAACATGTGAGCAAAAGGCCAGCAAAAGGCCA
GGAACCGTAAAAAGGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACCTGTCCGCCTTTCTCCCTTC
GGGAAGCGTGGCGCTTTCTCATAGCTCACGCTGTAGGTATCTCAGTTCGGTGTAGGTCGTTCGCTCCAA
GCTGGGCTGTGTGCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGATTAGCAGAGCG
AGGTATGTAGGCGGTGCTACAGAGTTCTTGAAGTGGTGGCCTAACTACGGCTACACTAGAAGAACAGT
ATTTGGTATCTGCGCTCTGCTGAAGCCAGTTACCTTCGGAAAAAGAGTTGGTAGCTCTTGATCCGGCAA
ACAAACCACCGCTGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCAGTGGAACGAAAACTCACGTTAAGGG
ATTTTGGTCATGAGATTATCAAAAAGGATCTTCACCTAGATCCTTTTAAATTAAAAATGAAGTTTTAAA
TCAATCTAAAGTATATATGAGTAAACTTGGTCTGACAGTTACCAATGCTTAATCAGTGAGGCACCTATC
TCAGCGATCTGTCTATTTCGTTCATCCATAGTTGCCTGACTCCCCGTCGTGTAGATAACTACGATACGG
GAGGGCTTACCATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTTATCCGCCTCCA
TCCAGTCTATTAATTGTTGCCGGGAAGCTAGAGTAAGTAGTTCGCCAGTTAATAGTTTGCGCAACGTTG
TTGCCATTGCTACAGGCATCGTGGTGTCACGCTCGTCGTTTGGTATGGCTTCATTCAGCTCCGGTTCCC
AACGATCAAGGCGAGTTACATGATCCCCCATGTTGTGCAAAAAAGCGGTTAGCTCCTTCGGTCCTCCG
ATCGTTGTCAGAAGTAAGTTGGCCGCAGTGTTATCACTCATGGTTATGGCAGCACTGCATAATTCTCTT
ACTGTCATGCCATCCGTAAGATGCTTTTCTGTGACTGGTGAGTACTCAACCAAGTCATTCTGAGAATAG
TGTATGCGGCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAAGTGCCAC
CTGACGTCTAAGAAACCATTATTATCATGACATTAACCTATAAAAATAGGCGTATCACGAGGCCCTTT
CGTCTCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGGTGTC
GGGGCTGGCTTAACTATGCGGCATCAGAGCAGATTGTACTGAGAGTGCACCATATGCGGTGTGAAATA
CCGCACAGATGCGTAAGGAGAAAATACCGCATCAGGCGATTCCAACATCCAATAAATCATACAGGCA
AGGCAAAGAATTAGCAAAATTAAGCAATAAAGCCTCAGAGCATAAAGCTAAATCGGTTGTACCAAAA
ACATTATGACCCTGTAATACTTTTGCGGGAGAAGCCTTTATTTCAACGCAAGGATAAAAATTTTTAGAA
CCCTCATATATTTTAAATGCAATGCCTGAGTAATGTGTAGGTAAAGATTCAAACGGGTGAGAAAGGCC
GGAGACAGTCAAATCACCATCAATATGATATTCAACCGTTCTAGCTGATAAATTCATGCCGGAGAGGG
TAGCTATTTTTGAGAGGTCTCTACAAAGGCTATCAGGTCATTGCCTGAGAGTCTGGAGCAAACAAGAG
AATCGATGAACGGTAATCGTAAAACTAGCATGTCAATCATATGTACCCCGGTTGATAATCAGAAAAGC
CCCAAAAACAGGAAGATTGTATAAGCAAATATTTAAATTGTAAGCGTTAATATTTTGTTAAAATTCGC
GTTAAATTTTTGTTAAATCAGCTCATTTTTTAACCAATAGGCCGAAATCGGCAAAATCCCTTATAAATC
AAAAGAATAGACCGAGATAGGGTTGAGTGTTGTTCCAGTTTGGAACAAGAGTCCACTATTAAAGAAC
GTGGACTCCAACGTCAAAGGGCGAAAAACCGTCTATCAGGGCGATGGCCCACTACGTGAACCATCAC
CCTAATCAAGTTTTTTGGGGTCGAGGTGCCGTAAAGCACTAAATCGGAACCCTAAAGGGAGCCCCCGA
TTTAGAGCTTGACGGGGAAAGCCGGCGAACGTGGCGAGAAAGGAAGGGAAGAAAGCGAAAGGAGCG
GGCGCTAGGGCGCTGGCAAGTGTAGCGGTCACGCTGCGCGTAACCACCACACCCGCCGCGCTTAATGC
GCCGCTACAGGGCGCGTACTATGGTTGCTTTGACGAGCACGTATAACGTGCTTTCCTCGTTAGAATCAG
AGCGGGAGCTAAACAGGAGGCCGATTAAAGGGATTTTAGACAGGAACGGTACGCCAGAATCCTGAGA
AGTGTTTTTATAATCAGTGAGGCCACCGAGTAAAAGAGTCTGTCCATCACGCAAATTAACCGTTGTCG
CAATACTTCTTTGATTAGTAATAACATCACTTGCCTGAGTAGAAGAACTCAAACTATCGGCCTTGCTGG
TAATATCCAGAACAATATTACCGCCAGCCATTGCAACGGAATCGCCATTCGCCATTCAGGCTGCGCAA
CTGTTGGGAAGGGCGATCGGTGCGGGCCTCTTCGCTATTACGCCAGCTGCGCGCTCGCTCGCTCACTG
AGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTAGGAAGCTTTCGTTACATAACTTACGG
TAAATGGCCCGCCTGGCTGACCGCCCAACGACCCCCGGACTCACGGGGATTTCCAAGTCTCCACCCCA
TTGACGTCAATGGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAGAGCTCGTTTAGT
GAACCGCTCGACCGGTCGCCATGAGCGGGGGGGGCAGGGCGCTGGCCGCGCTGCTGCTGGCCGCGTC
CGTGCTGAGCGCCGCGCTGCTGGCCCCCGGCGGCTCTTCGGGGCGCGATGCCCAGGCCGCGCCGCCAC
GAGACTTAGACAAAAAAAGACATGCAGAGCTGAAGATGGATCAGGCTTTGCTACTCATCCATAATGA
ACTTCTCTGGACCAACTTGACCGTCTACTGGAAATCTGAATGCTGTTATCACTGCTTGTTTCAGGTTCT
GGTAAACGTTCCTCAGAGTCCAAAAGCAGGGAAGCCTAGTGCTGCAGCTGCCTCTGTCAGCACCCAGC
ACGGATCTATCCTGCAGCTGAACGACACCTTGGAAGAGAAAGAAGTTTGTAGGTTGGAATACAGATTT
GGAGAATTTGGAAACTATTCTCTCTTGGTAAAGAACATCCATAATGGAGTTAGTGAAATTGCCTGTGA
CCTGGCTGTGAACGAGGATCCAGTTGATAGTAACCTTCCTGTGAGCATTGCATTCCTTATTGGTCTTGC
TGTCATCATTGTGATATCCTTTCTGAGGCTCTTGTTGAGTTTGGATGACTTTAACAATTGGATTTCTAAA
GCCATAAGTTCTCGAGAAACTGATCGCCTCATCAATTCTGAGCTGGGATCTCCCAGCAGGACAGACCC
TCTCGATGGTGATGTTCAGCCAGCAACGTGGCGTCTATCTGCCCTGCCGCCCCGCCTCCGCAGCGTGGA
CACCTTCAGGGGGATTGCTCTTATACTCATGGTCTTTGTCAATTATGGAGGAGGAAAATATTGGTACTT
CAAACATGCAAGTTGGAATGGGCTGACAGTGGCTGACCTCGTGTTCCCGTGGTTTGTATTTATTATGGG
ATCTTCCATTTTTCTATCGATGACTTCTATACTGCAACGGGGGTGTTCAAAATTCAGATTGCTGGGGAA
GATTGCATGGAGGAGTTTCCTGTTAATCTGCATAGGAATTATCATTGTGAATCCCAATTATTGCCTTGG
TCCATTGTCTTGGGACAAGGTGCGCATTCCTGGTGTGCTGCAGCGATTGGGAGTGACATACTTTGTGGT
TGCTGTGTTGGAGCTCCTCTTTGCTAAACCTGTGCCTGAACATTGTGCCTCGGAGAGGAGCTGCCTTTC
TCTTCGAGACATCACGTCCAGCTGGCCCCAGTGGCTGCTCATCCTGGTGCTGGAAGGCCTGTGGCTGG
GCTTGACATTCCTCCTGCCAGTCCCTGGGTGCCCTACTGGTTATCTTGGTCCTGGGGGCATTGGAGATT
TTGGCAAGTATCCAAATTGCACTGGAGGAGCTGCAGGCTACATCGACCGCCTGCTGCTGGGAGACGAT
CACCTTTACCAGCACCCATCTTCTGCTGTACTTTACCACACCGAGGTGGCCTATGACCCCGAGGGCATC
CTGGGCACCATCAACTCCATCGTGATGGCCTTTTTAGGAGTTCAGGCAGGAAAAATACTATTGTATTAC
AAGGCTCGGACCAAAGACATCCTGATTCGATTCACTGCTTGGTGTTGTATTCTTGGGCTCATTTCTGTT
GCTCTGACGAAGGTTTCTGAAAATGAAGGCTTTATTCCAGTAAACAAAAATCTCTGGTCCCTTTCGTAT
GTCACTACGCTCAGTTCTTTTGCCTTCTTCATCCTGCTGGTCCTGTACCCAGTTGTGGATGTGAAGGGG
CTGTGGACAGGAACCCCATTCTTTTATCCAGGAATGAATTCCATTCTGGTATATGTCGGCCACGAGGTG
TTTGAGAACTACTTCCCCTTTCAGTGGAAGCTGAAGGACAACCAGTCCCACAAGGAGCACCTGACTCA
GAACATCGTCGCCACTGCCCTCTGGGTGCTCATTGCCTACATCCTCTATAGAAAGAAGATTTTTTGGAA
AATCTGACCATCAGATCTAAATAAAATACGAAATGTTAAACCCCACTCCCTCTCTGCGCGCTCGCTCGC
TCACTGAGGCCGGGCGACCAAAGGTCGCCCGACGCCCGGGCTTTGCCCGGGCGGCCTCAGTGAGCGA
GCGAGCGCGCAGCTGCTG
ptrsk-CMV-hSMN1^{ZC} (SEQ ID NO: 6)
CAGCAGCTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGGGTTTAAACCTAAAAAACCTCCC
ACACCTCCCCCTGAACCTGAAACATAAAATGAATGCAATTGTTGTTGTTAACTTGTTTATTGCAGCTTA
TAATGGTTACAAATAAAGCAATAGCATCACAAATTTCACAAATAAAGCATTTTTTTCACTGCATTCTAG
TTGTGGTTTGTCCAAACTCATCAATGTATCTTATCATGTCTGAGATCTACAGGCGGAGTCCAGGCGGCA
GGGTTTAATTTAAGGAATGTGAGCACCTTCCTTCTTTTTGATTTTGTCTGAAACCCATATAATAGCCAG
TATGATAGCCACTCATGTACCATGAAATTAACATACTTCCCAAAGCATCAGCATCATCAAGAGAATCT
GGACATATGGGAGGTGGTGGGGGAATTATTGGTGGTCCAGAAGGAAATGGAGGCAGCCAGCATGATA
GTAAGTGGGGTGGTGGTGGTGGCGGTGGCGGTGGTGGGCCATTGAATTTTAGACCTGGCTTTCCTGGT
CCCAGTCTTGGCCCTGGCATGGGGGGTGGTGGAGGGAGAAAAGAGTTCCATGGAGCAGATTTGGGCTT
GATGTTATCTGATTTATTTCCAGGAGACCTGGAGTTCTCACTTTCATCTGTTGAAACTTGGCTTTCATTT
TCATTCTCTTGAGCATTTTGTTCTATATTATTAGCTACTTCACAGATTGGGGAAAGTAGATCGGACAGA
TTTTGCTCCTCTCTATTTCCATATCCAGTGTAAACCACAACACAGGTTTCTCTCTTAAAATCAATTGAAG
CAATGGTAGCTGGGTAAATGCAACCGTCTTCTGACCAAATGGCAGAACATTTGTCCCCAACTTTCCACT
GTTGTAAGGAAGCTGCAGTATTCTTCTTTTGGCTTTTATTCTTCTTAGCAGGTTTTCTTTTAGGTGTGGT
TTTTGGTTTACCCGAAGTTTCACAAATGTCACCATTCTTTAGAGCATGCTTAAATGAAGCCACAGCTTT
ATCATATGCTTTTATCAGTGCTGTATCATCCCAAATGTCAGAATCATCGCTCTGGCCTGTGCCGCGCCG
GAACAGCACGGAATCCTCCTGCTCCGGGACGCCGCCACCACTGCCGCCGCTGCTCATCGCCATAccggT
CCGGAGGCTGGATCGGTCCCGGTGTCTTCTATGGAGGTCAAAACAGCGTGGATGGCGTCTCCAGGCGA
TCTGACGGTTCACTAAACGAGCTCTGCTTATATAGACCTCCCACCGTACACGCCTACCGCCCATTTGCG
TCAATGGGGCGGAGTTGTTACGACATTTTGGAAAGTCCCGTTGATTTTGGTGCCAAAACAAACTCCCA
TTGACGTCAATGGGGTGGAGACTTGGAAATCCCCGTGAGTCAAACCGCTATCCACGCCCATTGATGTA
CTGCCAAAACCGCATCACCATGGTAATAGCGATGACTAATACGTAGATGTACTGCCAAGTAGGAAAGT
CCCATAAGGTCATGTACTGGGCATAATGCCAGGCGGGCCATTTACCGTCATTGACGTCAATAGGGGGC
GTACTTGGCATATGATACACTTGATGTACTGCCAAGTGGGCAGTTTACCGTAAATACTCCACCCATTGA
CGTCAATGGAAAGTCCCTATTGGCGTTACTATGGGAACATACGTCATTATTGACGTCAATGGGCGGGG
GTCGTTGGGCGGTCAGCCAGGCGGGCCATTTACCGTAAGTTATGTAACGAagcttCCTAGGAACCCCTAG
TGATGGAGTTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGCTGGCGTAATAGCGAAG
AGGCCCGCACCGATCGCCCTTCCCAACAGTTGCGCAGCCTGAATGGCGAATGGcgattCCGTTGCAATGG
CTGGCGGTAATATTGTTCTGGATATTACCAGCAAGGCCGATAGTTTGAGTTCTTCTACTCAGGCAAGTG
ATGTTATTACTAATCAAAGAAGTATTGCGACAACGGTTAATTTGCGTGATGGACAGACTCTTTTACTCG
GTGGCCTCACTGATTATAAAAACACTTCTCAGGATTCTGGCGTACCGTTCCTGTCTAAAATCCCTTTAA
TCGGCCTCCTGTTTAGCTCCCGCTCTGATTCTAACGAGGAAAGCACGTTATACGTGCTCGTCAAAGCAA
CCATAGTACGCGCCCTGTAGCGGCGCATTAAGCGCGGCGGGTGTGGTGGTTACGCGCAGCGTGACCGC
TACACTTGCCAGCGCCCTAGCGCCCGCTCCTTTCGCTTTCTTCCCTTCCTTTCTCGCCACGTTCGCCGGC
TTTCCCCGTCAAGCTCTAAATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAACACTCAACCCTATC
TCGGTCTATTCTTTTGATTTATAAGGGATTTTGCCGATTTCGGCCTATTGGTTAAAAAATGAGCTGATTT
AACAAAAATTTAACGCGAATTTTAACAAAATATTAACGcTTACAATTTAAATATTTGCTTATACAATCT
TCCTGTTTTTGGGGCTTTTCTGATTATCAACCGGGGTACATATGATTGACATGCTAGTTTTACGATTACC
GTTCATCGATTCTCTTGTTTGCTCCAGACTCTCAGGCAATGACCTGATAGCCTTTGTAGAGACCTCTCA
AAAATAGCTACCCTCTCCGGCATGAATTTATCAGCTAGAACGGTTGAATATCATATTGATGGTGATTTG
ACTGTCTCCGGCCTTTCTCACCCGTTTGAATCTTTACCTACACATTACTCAGGCATTGCATTTAAAATAT
ATGAGGGTTCTAAAAATTTTTATCCTTGCGTTGAAATAAAGGCTTCTCCCGCAAAAGTATTACAGGGTC
ATAATGTTTTTGGTACAACCGATTTAGCTTTATGCTCTGAGGCTTTATTGCTTAATTTTGCTAATTCTTT
GCCTTGCCTGTATGATTTATTGGATGTTGGAATcgCCTGATGCGGTATTTTCTCCTTACGCATCTGTGCG
GTATTTCACACCGCATATGGTGCACTCTCAGTACAATCTGCTCTGATGCCGCATAGTTAAGCCAGCCCC
GACACCCGCCAACACCCGCTGACGCGCCCTGACGGGCTTGTCTGCTCCCGGCATCCGCTTACAGACAA
GCTGTGACCGTCTCCGGGAGCTGCATGTGTCAGAGGTTTTCACCGTCATCACCGAAACGCGCGAGACG
AAAGGGCCTCGTGATACGCCTATTTTTATAGGTTAATGTCATGATAATAATGGTTTCTTAGACGTCAGG
TGGCACTTTTCGGGGAAATGTGCGCGGAACCCCTATTTGTTTATTTTTCTAAATACATTCAAATATGTA
TCCGCTCATGAGACAATAACCCTGATAAATGCTTCAATAATATTGAAAAAGGAAGAGTATGAGTATTC
AACATTTCCGTGTCGCCCTTATTCCCTTTTTTGCGGCATTTTGCCTTCCTGTTTTTGCTCACCCAGAAAC
GCTGGTGAAAGTAAAAGATGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATGATGAGCACTTTTAAAGTTC
TGCTATGTGGCGCGGTATTATCCCGTATTGACGCCGGGCAAGAGCAACTCGGTCGCCGCATACACTAT
TCTCAGAATGACTTGGTTGAGTACTCACCAGTCACAGAAAAGCATCTTACGGATGGCATGACAGTAAG
AGAATTATGCAGTGCTGCCATAACCATGAGTGATAACACTGCGGCCAACTTACTTCTGACAACGATCG
GAGGACCGAAGGAGCTAACCGCTTTTTTGCACAACATGGGGGATCATGTAACTCGCCTTGATCGTTGG
GAACCGGAGCTGAATGAAGCCATACCAAACGACGAGCGTGACACCACGATGCCTGTAGCAATGGCAA
CAACGTTGCGCAAACTATTAACTGGCGAACTACTTACTCTAGCTTCCCGGCAACAATTAATAGACTGG
ATGGAGGCGGATAAAGTTGCAGGACCACTTCTGCGCTCGGCCCTTCCGGCTGGCTGGTTTATTGCTGAT
AAATCTGGAGCCGGTGAGCGTGGGTCTCGCGGTATCATTGCAGCACTGGGGCCAGATGGTAAGCCCTC
CCGTATCGTAGTTATCTACACGACGGGGAGTCAGGCAACTATGGATGAACGAAATAGACAGATCGCTG
AGATAGGTGCCTCACTGATTAAGCATTGGTAACTGTCAGACCAAGTTTACTCATATATACTTTAGATTG
ATTTAAAACTTCATTTTTAATTTAAAAGGATCTAGGTGAAGATCCTTTTTGATAATCTCATGACCAAAA
TCCCTTAACGTGAGTTTTCGTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGAGCGCAGATACCAAAT
ACTGTtCTTCTAGTGTAGCCGTAGTTAGGCCACCACTTCAAGAACTCTGTAGCACCGCCTACATACCTC
GCTCTGCTAATCCTGTTACCAGTGGCTGCTGCCAGTGGCGATAAGTCGTGTCTTACCGGGTTGGACTCA
AGACGATAGTTACCGGATAAGGCGCAGCGGTCGGGCTGAACGGGGGGTTCGTGCACACAGCCCAGCT
TGGAGCGAACGACCTACACCGAACTGAGATACCTACAGCGTGAGCTATGAGAAAGCGCCACGCTTCC
CGAAGGGAGAAAGGCGGACAGGTATCCGGTAAGCGGCAGGGTCGGAACAGGAGAGCGCACGAGGGA
GCTTCCAGGGGGAAACGCCTGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCAACGCGGCCTTTTTACGGT
TCCTGGCCTTTTGCTGGCCTTTTGCTCACATGTTCTTTCCTGCGTTATCCCCTGATTCTGTGGATAACCG
TATTACCGCCTTTGAGTGAGCTGATACCGCTCGCCGCAGCCGAACGACCGAGCGCAGCGAGTCAGTGA
GCGAGGAAGCGGAAGAGCGCCCAATACGCAAACCGCCTCTCCCCGCGCGTTGGCCGATTCATTAATG
ptrsk-CMV-hSMN1 (SEQ ID NO: 7)
CAGCAGCTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGGGTTTAAACCTAAAAAACCTCCC
ACACCTCCCCCTGAACCTGAAACATAAAATGAATGCAATTGTTGTTGTTAACTTGTTTATTGCAGCTTA
TAATGGTTACAAATAAAGCAATAGCATCACAAATTTCACAAATAAAGCATTTTTTTCACTGCATTCTAG
TTGTGGTTTGTCCAAACTCATCAATGTATCTTATCATGTCTGAGATCTTTAATTTAAGGAATGTGAGCA
CCTTCCTTCTTTTTGATTTTGTCTGAAACCCATATAATAGCCAGTATGATAGCCACTCATGTACCATGA
AATTAACATACTTCCCAAAGCATCAGCATCATCAAGAGAATCTGGACATATGGGAGGTGGTGGGGGA
ATTATTGGTGGTCCAGAAGGAAATGGAGGCAGCCAGCATGATAGTAAGTGGGGTGGTGGTGGTGGCG
GTGGCGGTGGTGGGCCATTGAATTTTAGACCTGGCTTTCCTGGTCCCAGTCTTGGCCCTGGCATGGGGG
GTGGTGGAGGGAGAAAAGAGTTCCATGGAGCAGATTTGGGCTTGATGTTATCTGATTTATTTCCAGGA
GACCTGGAGTTCTCACTTTCATCTGTTGAAACTTGGCTTTCATTTTCATTCTCTTGAGCATTTTGTTCTAT
ATTATTAGCTACTTCACAGATTGGGGAAAGTAGATCGGACAGATTTTGCTCCTCTCTATTTCCATATCC
AGTGTAAACCACAACACAGGTTTCTCTCTTAAAATCAATTGAAGCAATGGTAGCTGGGTAAATGCAAC
CGTCTTCTGACCAAATGGCAGAACATTTGTCCCCAACTTTCCACTGTTGTAAGGAAGCTGCAGTATTCT
TCTTTTGGCTTTTATTCTTCTTAGCAGGTTTTCTTTTAGGTGTGGTTTTTGGTTTACCCGAAGTTTCACAA
ATGTCACCATTCTTTAGAGCATGCTTAAATGAAGCCACAGCTTTATCATATGCTTTTATCAGTGCTGTA
TCATCCCAAATGTCAGAATCATCGCTCTGGCCTGTGCCGCGCCGGAACAGCACGGAATCCTCCTGCTC
CGGGACGCCGCCACCACTGCCGCCGCTGCTCATCGCCATACCGGTCCGGAGGCTGGATCGGTCCCGGT
GTCTTCTATGGAGGTCAAAACAGCGTGGATGGCGTCTCCAGGCGATCTGACGGTTCACTAAACGAGCT
CTGCTTATATAGACCTCCCACCGTACACGCCTACCGCCCATTTGCGTCAATGGGGCGGAGTTGTTACGA
CATTTTGGAAAGTCCCGTTGATTTTGGTGCCAAAACAAACTCCCATTGACGTCAATGGGGTGGAGACT
TGGAAATCCCCGTGAGTCAAACCGCTATCCACGCCCATTGATGTACTGCCAAAACCGCATCACCATGG
TAATAGCGATGACTAATACGTAGATGTACTGCCAAGTAGGAAAGTCCCATAAGGTCATGTACTGGGCA
TAATGCCAGGCGGGCCATTTACCGTCATTGACGTCAATAGGGGGCGTACTTGGCATATGATACACTTG
ATGTACTGCCAAGTGGGCAGTTTACCGTAAATACTCCACCCATTGACGTCAATGGAAAGTCCCTATTG
GCGTTACTATGGGAACATACGTCATTATTGACGTCAATGGGCGGGGGTCGTTGGGCGGTCAGCCAGGC
GGGCCATTTACCGTAAGTTATGTAACGAAGCTTCCTAGGAACCCCTAGTGATGGAGTTGGCCACTCCC
TCTCTGCGCGCTCGCTCGCTCACTGAGGCCGGGCGACCAAAGGTCGCCCGACGCCCGGGCTTTGCCCG
GGCGGCCTCAGTGAGCGAGCGAGCGCGCAGCTGGCGTAATAGCGAAGAGGCCCGCACCGATCGCCCT
TCCCAACAGTTGCGCAGCCTGAATGGCGAATGGCGATTCCGTTGCAATGGCTGGCGGTAATATTGTTC
TGGATATTACCAGCAAGGCCGATAGTTTGAGTTCTTCTACTCAGGCAAGTGATGTTATTACTAATCAAA
GAAGTATTGCGACAACGGTTAATTTGCGTGATGGACAGACTCTTTTACTCGGTGGCCTCACTGATTATA
AAAACACTTCTCAGGATTCTGGCGTACCGTTCCTGTCTAAAATCCCTTTAATCGGCCTCCTGTTTAGCT
CCCGCTCTGATTCTAACGAGGAAAGCACGTTATACGTGCTCGTCAAAGCAACCATAGTACGCGCCCTG
TAGCGGCGCATTAAGCGCGGCGGGTGTGGTGGTTACGCGCAGCGTGACCGCTACACTTGCCAGCGCCC
TAGCGCCCGCTCCTTTCGCTTTCTTCCCTTCCTTTCTCGCCACGTTCGCCGGCTTTCCCCGTCAAGCTCT
AAATCGGGGGCTCCCTTTAGGGTTCCGATTTAGTGCTTTACGGCACCTCGACCCCAAAAAACTTGATTA
GGGTGATGGTTCACGTAGTGGGCCATCGCCCTGATAGACGGTTTTTCGCCCTTTGACGTTGGAGTCCAC
GTTCTTTAATAGTGGACTCTTGTTCCAAACTGGAACAACACTCAACCCTATCTCGGTCTATTCTTTTGAT
TTATAAGGGATTTTGCCGATTTCGGCCTATTGGTTAAAAAATGAGCTGATTTAACAAAAATTTAACGCG
AATTTTAACAAAATATTAACGCTTACAATTTAAATATTTGCTTATACAATCTTCCTGTTTTTGGGGCTTT
TCTGATTATCAACCGGGGTACATATGATTGACATGCTAGTTTTACGATTACCGTTCATCGATTCTCTTGT
TTGCTCCAGACTCTCAGGCAATGACCTGATAGCCTTTGTAGAGACCTCTCAAAAATAGCTACCCTCTCC
GGCATGAATTTATCAGCTAGAACGGTTGAATATCATATTGATGGTGATTTGACTGTCTCCGGCCTTTCT
CACCCGTTTGAATCTTTACCTACACATTACTCAGGCATTGCATTTAAAATATATGAGGGTTCTAAAAAT
TTTTATCCTTGCGTTGAAATAAAGGCTTCTCCCGCAAAAGTATTACAGGGTCATAATGTTTTTGGTACA
ACCGATTTAGCTTTATGCTCTGAGGCTTTATTGCTTAATTTTGCTAATTCTTTGCCTTGCCTGTATGATTT
ATTGGATGTTGGAATCGCCTGATGCGGTATTTTCTCCTTACGCATCTGTGCGGTATTTCACACCGCATA
TGGTGCACTCTCAGTACAATCTGCTCTGATGCCGCATAGTTAAGCCAGCCCCGACACCCGCCAACACC
CGCTGACGCGCCCTGACGGGCTTGTCTGCTCCCGGCATCCGCTTACAGACAAGCTGTGACCGTCTCCG
GGAGCTGCATGTGTCAGAGGTTTTCACCGTCATCACCGAAACGCGCGAGACGAAAGGGCCTCGTGATA
CGCCTATTTTTATAGGTTAATGTCATGATAATAATGGTTTCTTAGACGTCAGGTGGCACTTTTCGGGGA
AATGTGCGCGGAACCCCTATTTGTTTATTTTTCTAAATACATTCAAATATGTATCCGCTCATGAGACAA
TAACCCTGATAAATGCTTCAATAATATTGAAAAAGGAAGAGTATGAGTATTCAACATTTCCGTGTCGC
CCTTATTCCCTTTTTTGCGGCATTTTGCCTTCCTGTTTTTGCTCACCCAGAAACGCTGGTGAAAGTAAAA
GATGCTGAAGATCAGTTGGGTGCACGAGTGGGTTACATCGAACTGGATCTCAACAGCGGTAAGATCCT
TGAGAGTTTTCGCCCCGAAGAACGTTTTCCAATGATGAGCACTTTTAAAGTTCTGCTATGTGGCGCGGT
ATTATCCCGTATTGACGCCGGGCAAGAGCAACTCGGTCGCCGCATACACTATTCTCAGAATGACTTGG
TTGAGTACTCACCAGTCACAGAAAAGCATCTTACGGATGGCATGACAGTAAGAGAATTATGCAGTGCT
GCCATAACCATGAGTGATAACACTGCGGCCAACTTACTTCTGACAACGATCGGAGGACCGAAGGAGCT
AACCGCTTTTTTGCACAACATGGGGGATCATGTAACTCGCCTTGATCGTTGGGAACCGGAGCTGAATG
AAGCCATACCAAACGACGAGCGTGACACCACGATGCCTGTAGCAATGGCAACAACGTTGCGCAAACT
ATTAACTGGCGAACTACTTACTCTAGCTTCCCGGCAACAATTAATAGACTGGATGGAGGCGGATAAAG
TTGCAGGACCACTTCTGCGCTCGGCCCTTCCGGCTGGCTGGTTTATTGCTGATAAATCTGGAGCCGGTG
AGCGTGGGTCTCGCGGTATCATTGCAGCACTGGGGCCAGATGGTAAGCCCTCCCGTATCGTAGTTATC
TACACGACGGGGAGTCAGGCAACTATGGATGAACGAAATAGACAGATCGCTGAGATAGGTGCCTCAC
TGATTAAGCATTGGTAACTGTCAGACCAAGTTTACTCATATATACTTTAGATTGATTTAAAACTTCATT
TTTAATTTAAAAGGATCTAGGTGAAGATCCTTTTTGATAATCTCATGACCAAAATCCCTTAACGTGAGT
TTTCGTTCCACTGAGCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTTGCCGGATCAAGAG
CTACCAACTCTTTTTCCGAAGGTAACTGGCTTCAGCAGAGCGCAGATACCAAATACTGTTCTTCTAGTG
TAGCCGTAGTTAGGCCACCACTTCAAGAACTCTGTAGCACCGCCTACATACCTCGCTCTGCTAATCCTG
TTACCAGTGGCTGCTGCCAGTGGCGATAAGTCGTGTCTTACCGGGTTGGACTCAAGACGATAGTTACC
GGATAAGGCGCAGCGGTCGGGCTGAACGGGGGGTTCGTGCACACAGCCCAGCTTGGAGCGAACGACC
TACACCGAACTGAGATACCTACAGCGTGAGCTATGAGAAAGCGCCACGCTTCCCGAAGGGAGAAAGG
CGGACAGGTATCCGGTAAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAGCGTCGATTTTTGTGATGCTC
GTCAGGGGGGCGGAGCCTATGGAAAAACGCCAGCAACGCGGCCTTTTTACGGTTCCTGGCCTTTTGCT
GGCCTTTTGCTCACATGTTCTTTCCTGCGTTATCCCCTGATTCTGTGGATAACCGTATTACCGCCTTTGA
GTGAGCTGATACCGCTCGCCGCAGCCGAACGACCGAGCGCAGCGAGTCAGTGAGCGAGGAAGCGGAA
GAGCGCCCAATACGCAAACCGCCTCTCCCCGCGCGTTGGCCGATTCATTAATG

1. An expression vector comprising a polynucleotide encoding a nucleic acid of interest operably linked to an extracellular vesicle-targeting zip code sequence.

2. The expression vector of claim 1, wherein the extracellular vesicle-targeting zip code sequence is linked to the 3′ end of the polynucleotide.

3. The expression vector of claim 1, wherein the polynucleotide is operably linked to a promoter and a poly (A) signal and the extracellular vesicle-targeting zip code sequence is located between the polynucleotide and the poly (A) signal.

4. The expression vector of claim 1, wherein the extracellular vesicle-targeting zip code sequence comprises the nucleotide sequence of SEQ ID NO:1.

5. (canceled)

6. The expression vector of claim 1, wherein the expression vector is a plasmid vector or a viral vector.

7. The expression vector of claim 6, wherein the expression vector is an adeno-associated virus vector.

8. The expression vector of claim 7, wherein the adeno-associated virus comprises a serotype selected from the group consisting of AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, and a chimeric AAV vector.

9. The expression vector of claim 7, wherein the expression vector is a self-complementary AAV vector.

10. The expression vector of claim 1, wherein the nucleic acid of interest encodes a therapeutic protein or a functional nucleic acid.

11. The expression vector of claim 10, wherein the therapeutic protein is a secreted protein.

12. The expression vector of claim 10, wherein the therapeutic protein is a non-secreted protein.

13. The expression vector of claim 10, wherein the therapeutic protein is heparan alpha-glucosaminide N-acetyltransferase (HGSNAT) or survival motor neuron 1 (SMN1).

14-15. (canceled)

16. A virus particle comprising the expression vector of claim 1.

17. The virus particle of claim 16, wherein the virus particle is an AAV particle, an adenovirus particle, a herpesvirus particle, or a baculovirus particle.

18. A pharmaceutical composition comprising the expression vector of claim 1 and a pharmaceutically acceptable carrier.

19. A method of delivering a nucleic acid of interest to bystander cells in a subject, comprising administering to the subject an effective amount of the expression vector of claim 1, thereby forming extracellular vesicles comprising the nucleic acid of interest and delivering the nucleic acid of interest to bystander cells.

20. A method of treating a disorder treatable by expression of a nucleic acid of interest in a subject in need thereof, comprising administering to the subject a therapeutically effective amount of the expression vector of claim 1, thereby treating the disorder.

21. The method of claim 20, wherein the nucleic acid of interest encodes a therapeutic protein or a functional nucleic acid.

22. The method of claim 21, wherein the therapeutic protein is a secreted protein.

23. The method of claim 21, wherein the therapeutic protein is a non-secreted protein.

24. The method of claim 21, wherein the therapeutic protein is HGSNAT and the disorder is mucopolysaccharidosis IIIC or the therapeutic protein is SMN1 and the disorder is spinal muscle atrophy.

25-27. (canceled)


SEQUENCE LISTING (7 sequences)

SEQ ID NO: 1 - DNA, 25 bp, synthetic construct
  source 1..25: mol_type = other DNA
accctgccgcctggactccgcctgt

SEQ ID NO: 2 - DNA, 5965 bp, synthetic construct
  source 1..5965: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaatgagcggggcgggcagggcgctggccgcgctgctgctggccgcgtccgtgctgagcgccgcgctgctggcccccggcggctcttcggggcgcgatgcccaggccgcgccgccacgagacttagacaaaaaaagacatgcagagctgaagatggatcaggctttgctactcatccataatgaacttctctggaccaacttgaccgtctactggaaatctgaatgctgttatcactgcttgtttcaggttctggtaaacgttcctcagagtccaaaagcagggaagcctagtgctgcagctgcctctgtcagcacccagcacggatctatcctgcagctgaacgacaccttggaagagaaagaagtttgtaggttggaatacagatttggagaatttggaaactattctctcttggtaaagaacatccataatggagttagtgaaattgcctgtgacctggctgtgaacgaggatccagttgatagtaaccttcctgtgagcattgcattccttattggtcttgctgtcatcattgtgatatcctttctgaggctcttgttgagtttggatgactttaacaattggatttctaaagccataagttctcgagaaactgatcgcctcatcaattctgagctgggatctcccagcaggacagaccctctcgatggtgatgttcagccagcaacgtggcgtctatctgccctgccgccccgcctccgcagcgtggacaccttcagggggattgctcttatactcatggtctttgtcaattatggaggaggaaaatattggtacttcaaacatgcaagttggaatgggctgacagtggctgacctcgtgttcccgtggtttgtatttattatgggatcttccatttttctatcgatgacttctatactgcaacgggggtgttcaaaattcagattgctggggaagattgcatggaggagtttcctgttaatctgcataggaattatcattgtgaatcccaattattgccttggtccattgtcttgggacaaggtgcgcattcctggtgtgctgcagcgattgggagtgacatactttgtggttgctgtgttggagctcctctttgctaaacctgtgcctgaacattgtgcctcggagaggagctgcctttctcttcgagacatcacgtccagctggccccagtggctgctcatcctggtgctggaaggcctgtggctgggcttgacattcctcctgccagtccctgggtgccctactggttatcttggtcctgggggcattggagattttggcaagtatccaaattgcactggaggagctgcaggctacatcgaccgcctgctgctgggagacgatcacctttaccagcacccatcttctgctgtactttaccacaccgaggtggcctatgaccccgagggcatcctgggcaccatcaactccatcgtgatggcctttttaggagttcaggcaggaaaaatactattgtattacaaggctcggaccaaagacatcctgattcgattcactgcttggtgttgtattcttgggctcatttctgttgctctgacgaaggtttctgaaaatgaaggctttattccagtaaacaaaaatctctggtccctttcgtatgtcactacgctcagttcttttgccttcttcatcctgctggtcctgtacccagttgtggatgtgaaggggctgtggacaggaaccccattcttttatccaggaatgaattccattctggtatatgtcggccacgaggtgtttgagaactacttcccctttcagtggaagctgaaggacaaccagtcccacaaggagcacctgactcagaacatcgtcgccactgccctctgggtgctcattgcctacatcctctatagaaagaagattttttggaaaatctgagtcgacaccctgccgcctggactccgcctgttagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggctgca

SEQ ID NO: 3 - DNA, 5940 bp, synthetic construct
  source 1..5940: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaatgagcggggcgggcagggcgctggccgcgctgctgctggccgcgtccgtgctgagcgccgcgctgctggcccccggcggctcttcggggcgcgatgcccaggccgcgccgccacgagacttagacaaaaaaagacatgcagagctgaagatggatcaggctttgctactcatccataatgaacttctctggaccaacttgaccgtctactggaaatctgaatgctgttatcactgcttgtttcaggttctggtaaacgttcctcagagtccaaaagcagggaagcctagtgctgcagctgcctctgtcagcacccagcacggatctatcctgcagctgaacgacaccttggaagagaaagaagtttgtaggttggaatacagatttggagaatttggaaactattctctcttggtaaagaacatccataatggagttagtgaaattgcctgtgacctggctgtgaacgaggatccagttgatagtaaccttcctgtgagcattgcattccttattggtcttgctgtcatcattgtgatatcctttctgaggctcttgttgagtttggatgactttaacaattggatttctaaagccataagttctcgagaaactgatcgcctcatcaattctgagctgggatctcccagcaggacagaccctctcgatggtgatgttcagccagcaacgtggcgtctatctgccctgccgccccgcctccgcagcgtggacaccttcagggggattgctcttatactcatggtctttgtcaattatggaggaggaaaatattggtacttcaaacatgcaagttggaatgggctgacagtggctgacctcgtgttcccgtggtttgtatttattatgggatcttccatttttctatcgatgacttctatactgcaacgggggtgttcaaaattcagattgctggggaagattgcatggaggagtttcctgttaatctgcataggaattatcattgtgaatcccaattattgccttggtccattgtcttgggacaaggtgcgcattcctggtgtgctgcagcgattgggagtgacatactttgtggttgctgtgttggagctcctctttgctaaacctgtgcctgaacattgtgcctcggagaggagctgcctttctcttcgagacatcacgtccagctggccccagtggctgctcatcctggtgctggaaggcctgtggctgggcttgacattcctcctgccagtccctgggtgccctactggttatcttggtcctgggggcattggagattttggcaagtatccaaattgcactggaggagctgcaggctacatcgaccgcctgctgctgggagacgatcacctttaccagcacccatcttctgctgtactttaccacaccgaggtggcctatgaccccgagggcatcctgggcaccatcaactccatcgtgatggcctttttaggagttcaggcaggaaaaatactattgtattacaaggctcggaccaaagacatcctgattcgattcactgcttggtgttgtattcttgggctcatttctgttgctctgacgaaggtttctgaaaatgaaggctttattccagtaaacaaaaatctctggtccctttcgtatgtcactacgctcagttcttttgccttcttcatcctgctggtcctgtacccagttgtggatgtgaaggggctgtggacaggaaccccattcttttatccaggaatgaattccattctggtatatgtcggccacgaggtgtttgagaactacttcccctttcagtggaagctgaaggacaaccagtcccacaaggagcacctgactcagaacatcgtcgccactgccctctgggtgctcattgcctacatcctctatagaaagaagattttttggaaaatctgagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagctggctgca

SEQ ID NO: 4 - DNA, 6055 bp, synthetic construct
  source 1..6055: mol_type = other DNA
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctttcgttacataacttacggtaaatggcccgcctggctgaccgcccaacgacccccggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgctcgaccggtcgccatgagcggggcgggcagggcgctggccgcgctgctgctggccgcgtccgtgctgagcgccgcgctgctggcccccggcggctcttcggggcgcgatgcccaggccgcgccgccacgagacttagacaaaaaaagacatgcagagctgaagatggatcaggctttgctactcatccataatgaacttctctggaccaacttgaccgtctactggaaatctgaatgctgttatcactgcttgtttcaggttctggtaaacgttcctcagagtccaaaagcagggaagcctagtgctgcagctgcctctgtcagcacccagcacggatctatcctgcagctgaacgacaccttggaagagaaagaagtttgtaggttggaatacagatttggagaatttggaaactattctctcttggtaaagaacatccataatggagttagtgaaattgcctgtgacctggctgtgaacgaggatccagttgatagtaaccttcctgtgagcattgcattccttattggtcttgctgtcatcattgtgatatcctttctgaggctcttgttgagtttggatgactttaacaattggatttctaaagccataagttctcgagaaactgatcgcctcatcaattctgagctgggatctcccagcaggacagaccctctcgatggtgatgttcagccagcaacgtggcgtctatctgccctgccgccccgcctccgcagcgtggacaccttcagggggattgctcttatactcatggtctttgtcaattatggaggaggaaaatattggtacttcaaacatgcaagttggaatgggctgacagtggctgacctcgtgttcccgtggtttgtatttattatgggatcttccatttttctatcgatgacttctatactgcaacgggggtgttcaaaattcagattgctggggaagattgcatggaggagtttcctgttaatctgcataggaattatcattgtgaatcccaattattgccttggtccattgtcttgggacaaggtgcgcattcctggtgtgctgcagcgattgggagtgacatactttgtggttgctgtgttggagctcctctttgctaaacctgtgcctgaacattgtgcctcggagaggagctgcctttctcttcgagacatcacgtccagctggccccagtggctgctcatcctggtgctggaaggcctgtggctgggcttgacattcctcctgccagtccctgggtgccctactggttatcttggtcctgggggcattggagattttggcaagtatccaaattgcactggaggagctgcaggctacatcgaccgcctgctgctgggagacgatcacctttaccagcacccatcttctgctgtactttaccacaccgaggtggcctatgaccccgagggcatcctgggcaccatcaactccatcgtgatggcctttttaggagttcaggcaggaaaaatactattgtattacaaggctcggaccaaagacatcctgattcgattcactgcttggtgttgtattcttgggctcatttctgttgctctgacgaaggtttctgaaaatgaaggctttattccagtaaacaaaaatctctggtccctttcgtatgtcactacgctcagttcttttgccttcttcatcctgctggtcctgtacccagttgtggatgtgaaggggctgtggacaggaaccccattcttttatccaggaatgaattccattctggtatatgtcggccacgaggtgtttgagaactacttcccctttcagtggaagctgaaggacaaccagtcccacaaggagcacctgactcagaacatcgtcgccactgccctctgggtgctcattgcctacatcctctatagaaagaagattttttggaaaatctgacgtcgacaccctgccgcctggactccgcctgtcatcagatctaaataaaatacgaaatgttaaaccccactccctctctgcgcgctcgctcgctcactgaggccgggcgaccaaaggtcgcccgacgcccgggctttgcccgggcggcctcagtgagcgagcgagcgcgcagctgctg

SEQ ID NO: 5 - DNA, 6024 bp, synthetic construct
  source 1..6024: mol_type = other DNA
cattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctttcgttacataacttacggtaaatggcccgcctggctgaccgcccaacgacccccggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgctcgaccggtcgccatgagcggggcgggcagggcgctggccgcgctgctgctggccgcgtccgtgctgagcgccgcgctgctggcccccggcggctcttcggggcgcgatgcccaggccgcgccgccacgagacttagacaaaaaaagacatgcagagctgaagatggatcaggctttgctactcatccataatgaacttctctggaccaacttgaccgtctactggaaatctgaatgctgttatcactgcttgtttcaggttctggtaaacgttcctcagagtccaaaagcagggaagcctagtgctgcagctgcctctgtcagcacccagcacggatctatcctgcagctgaacgacaccttggaagagaaagaagtttgtaggttggaatacagatttggagaatttggaaactattctctcttggtaaagaacatccataatggagttagtgaaattgcctgtgacctggctgtgaacgaggatccagttgatagtaaccttcctgtgagcattgcattccttattggtcttgctgtcatcattgtgatatcctttctgaggctcttgttgagtttggatgactttaacaattggatttctaaagccataagttctcgagaaactgatcgcctcatcaattctgagctgggatctcccagcaggacagaccctctcgatggtgatgttcagccagcaacgtggcgtctatctgccctgccgccccgcctccgcagcgtggacaccttcagggggattgctcttatactcatggtctttgtcaattatggaggaggaaaatattggtacttcaaacatgcaagttggaatgggctgacagtggctgacctcgtgttcccgtggtttgtatttattatgggatcttccatttttctatcgatgacttctatactgcaacgggggtgttcaaaattcagattgctggggaagattgcatggaggagtttcctgttaatctgcataggaattatcattgtgaatcccaattattgccttggtccattgtcttgggacaaggtgcgcattcctggtgtgctgcagcgattgggagtgacatactttgtggttgctgtgttggagctcctctttgctaaacctgtgcctgaacattgtgcctcggagaggagctgcctttctcttcgagacatcacgtccagctggccccagtggctgctcatcctggtgctggaaggcctgtggctgggcttgacattcctcctgccagtccctgggtgccctactggttatcttggtcctgggggcattggagattttggcaagtatccaaattgcactggaggagctgcaggctacatcgaccgcctgctgctgggagacgatcacctttaccagcacccatcttctgctgtactttaccacaccgaggtggcctatgaccccgagggcatcctgggcaccatcaactccatcgtgatggcctttttaggagttcaggcaggaaaaatactattgtattacaaggctcggaccaaagacatcctgattcgattcactgcttggtgttgtattcttgggctcatttctgttgctctgacgaaggtttctgaaaatgaaggctttattccagtaaacaaaaatctctggtccctttcgtatgtcactacgctcagttcttttgccttcttcatcctgctggtcctgtacccagttgtggatgtgaaggggctgtggacaggaaccccattcttttatccaggaatgaattccattctggtatatgtcggccacgaggtgtttgagaactacttcccctttcagtggaagctgaaggacaaccagtcccacaaggagcacctgactcagaacatcgtcgccactgccctctgggtgctcattgcctacatcctctatagaaagaagattttttggaaaatctgaccatcagatctaaataaaatacgaaatgttaaaccccactccctctctgcgcgctcgctcgctcactgaggccgggcgaccaaaggtcgcccgacgcccgggctttgcccgggcggcctcagtgagcgagcgagcgcgcagctgctg

SEQ ID NO: 6 - DNA, 5559 bp, synthetic construct
  source 1..5559: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggggtttaaacctaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgagatctacaggcggagtccaggcggcagggtttaatttaaggaatgtgagcaccttccttctttttgattttgtctgaaacccatataatagccagtatgatagccactcatgtaccatgaaattaacatacttcccaaagcatcagcatcatcaagagaatctggacatatgggaggtggtgggggaattattggtggtccagaaggaaatggaggcagccagcatgatagtaagtggggtggtggtggtggcggtggcggtggtgggccattgaattttagacctggctttcctggtcccagtcttggccctggcatggggggtggtggagggagaaaagagttccatggagcagatttgggcttgatgttatctgatttatttccaggagacctggagttctcactttcatctgttgaaacttggctttcattttcattctcttgagcattttgttctatattattagctacttcacagattggggaaagtagatcggacagattttgctcctctctatttccatatccagtgtaaaccacaacacaggtttctctcttaaaatcaattgaagcaatggtagctgggtaaatgcaaccgtcttctgaccaaatggcagaacatttgtccccaactttccactgttgtaaggaagctgcagtattcttcttttggcttttattcttcttagcaggttttcttttaggtgtggtttttggtttacccgaagtttcacaaatgtcaccattctttagagcatgcttaaatgaagccacagctttatcatatgcttttatcagtgctgtatcatcccaaatgtcagaatcatcgctctggcctgtgccgcgccggaacagcacggaatcctcctgctccgggacgccgccaccactgccgccgctgctcatcgccataccggtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgaagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 7 - DNA, 5534 bp, synthetic construct
  source 1..5534: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggggtttaaacctaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgagatctttaatttaaggaatgtgagcaccttccttctttttgattttgtctgaaacccatataatagccagtatgatagccactcatgtaccatgaaattaacatacttcccaaagcatcagcatcatcaagagaatctggacatatgggaggtggtgggggaattattggtggtccagaaggaaatggaggcagccagcatgatagtaagtggggtggtggtggtggcggtggcggtggtgggccattgaattttagacctggctttcctggtcccagtcttggccctggcatggggggtggtggagggagaaaagagttccatggagcagatttgggcttgatgttatctgatttatttccaggagacctggagttctcactttcatctgttgaaacttggctttcattttcattctcttgagcattttgttctatattattagctacttcacagattggggaaagtagatcggacagattttgctcctctctatttccatatccagtgtaaaccacaacacaggtttctctcttaaaatcaattgaagcaatggtagctgggtaaatgcaaccgtcttctgaccaaatggcagaacatttgtccccaactttccactgttgtaaggaagctgcagtattcttcttttggcttttattcttcttagcaggttttcttttaggtgtggtttttggtttacccgaagtttcacaaatgtcaccattctttagagcatgcttaaatgaagccacagctttatcatatgcttttatcagtgctgtatcatcccaaatgtcagaatcatcgctctggcctgtgccgcgccggaacagcacggaatcctcctgctccgggacgccgccaccactgccgccgctgctcatcgccataccggtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgaagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg